SEQUENCE LISTING (6 sequences)

SEQ ID NO: 1 - DNA, 20 bp, Artificial Sequence
  PCR primer
gcggttaccctatccttttt

SEQ ID NO: 2 - DNA, 19 bp, Artificial Sequence
  PCR primer
gcagcaactttggcatttc

SEQ ID NO: 3 - DNA, 20 bp, Artificial Sequence
  PCR primer
ggcctgccaagctaatgaga

SEQ ID NO: 4 - DNA, 21 bp, Artificial Sequence
  PCR primer
gagcagctccatcctctggtt

SEQ ID NO: 5 - DNA, 21 bp, Artificial Sequence
  PCR primer
gtagtttcgtggatgccacag

SEQ ID NO: 6 - DNA, 20 bp, Artificial Sequence
  PCR primer
gagctacgagctgcctgacg

(19) United States
(12) Patent Application Publication
(10) Pub. No.: US 2015/0322001 A1
(43) Pub. Date: Nov. 12, 2015

(54) OMEGA-3 ANALOGUES

(71) Applicant: The University of Sydney, The university of Sydney (AU)

(72) Inventors: Michael MURRAY, The University of Sydney (AU); Tristan RAWLING, The University of Sydney (AU); Colin DUNSTAN, The University of Sydney (AU); Pei Hong CUI, The University of Sydney (AU)

(21) Appl. No.: 14/424,607

(22) PCT Filed: Nov. 21, 2013
(86) PCT No.: PCT/AU2013/001341

§ 371 Date: Feb. 27, 2015

(30) Foreign Application Priority Data
Nov. 21, 2012 (AU) 2012905120
Jul. 1, 2013 (AU) 2013902467

(51) Int. Cl. C07C 275/26; C07C 275/30; C07C 275/34; C07C 275/24; C07C 275/28

(52) U.S. Cl. CPC C07C 275/26; C07C 275/24; C07C 275/28; C07C 275/34; C07C 275/30; C07C 2101/14; C07C 2103/74

(57) ABSTRACT

The present invention relates to new fatty acid analogues and to their use in cancer therapy, including antimetastatic therapy.

### FIELD OF THE INVENTION

The present invention relates to new fatty acid analogues and to cancer therapy, including antimetastatic therapy.

### BACKGROUND OF THE INVENTION

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

The two major classes of dietary poly-unsaturated fatty acids (PUFAs) are the omega-3 and omega-6 PUFAs, typified by eicosapentaenoic acid (EPA) and arachidonic acid (AA), respectively. These PUFAs are structurally analogous, except that EPA has an additional olefinic bond between carbons 17 and 18 that is absent in AA.

High dietary intake of omega-6 PUFAs has been linked to an increased risk for prostate and other cancers, whereas omega-3 PUFA intake decreases risk (Berquin et al. 2011). However, anticancer strategies based on altered dietary regimen are unrealistic because of low patient compliance.

In the cell, both omega-3 and omega-6 PUFAs undergo biotransformation by cytochrome P450 (CYP), lipoxygenase and cyclooxygenase enzymes, which generate parallel series of eicosanoid metabolites with distinct biological actions, and mediate most of the cellular effects of PUFAs. Cyclooxygenases give rise to prostaglandins, lipoxygenases produce leukotrienes and CYPs generate PUFA epoxides.

Four enantiomeric monoepoxides (or EETs) are formed by CYP oxidation at each of the 5,6-, 8,9-, 11,12- and 14,15-olefinic double bonds of the omega-6 PUFA AA (Chen et al. 1998). In the case of the omega-3 PUFA EPA, CYPs also epoxygenate the fifth olefinic bond at C17-18, as well as the other four double bonds.

While dietary C17,18 omega-3 PUFA epoxides are understood to provide decreased risk of cancer, they are not produced in sufficient amounts in the body to have a therapeutic effect, and their duration of action is limited by the enzyme cytosolic epoxide hydrolase (cEH), which mediates their hydration to inactive diols (Inceoglue et al. 2007).

US 2008/0146663 and US 2008/0153889 relate to compounds that mimic epoxyeicosatrienoic acids (by the use of an ether group), and to the use of the compounds for the treatment of renal or cardiovascular diseases. Similar analogues are discussed in US 2008/0095711. WO 2011/066414 relates to omega-6 (specifically AA) analogues, and their use in analgesic treatment. Other omega-3 analogues are discussed in WO 2010/081683.

New anti-metastatic therapies, including therapies that target various stages of metastasis are required.

### SUMMARY OF THE INVENTION

The present invention relates to a compound of formula (I):

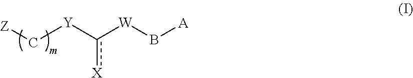

wherein

A is selected from OR¹, C(O)R¹, C(O)OR¹, C(O)NR¹R², OP(O)(OR¹)₂, C(O)OP(O)(OR¹)₂, P(OR¹)₃, C(O)OP(OR¹)₃, C(O)P(OR¹)₃, OS(O)(OR¹)₂, C(O)S(O)(OR¹)₂, OS(O)₂(OR¹), C(O)S(O)₂(OR¹), OSR¹, C(O)SR¹, OSR¹R², C(O)SR¹R², cycloalkyl, heterocycloalkyl and heteroaryl;

B is a hydrocarbon chain containing from 7 to 25 carbon atoms, wherein the hydrocarbon chain is saturated, branched or unbranched, and optionally includes one or more heteroatoms selected from O, N and S;

W and Y are selected from CH₂, O and NR¹, wherein W may form a 5- or 6-membered cycloalkyl or heterocycloalkyl ring with X and B;

X is selected from CH₂, O, NR¹ and S;

C is CH₂;

m is 0, 1 or 2;

Z is selected from alkyl, heteroalkyl, alkenyl, alkynyl, cycloalkyl, heterocycloalkyl, aryl and heteroaryl, which groups are optionally substituted,

wherein R¹ and R² are independently selected from H, OH, alkyl, heteroalkyl, alkenyl, alkynyl, cycloalkyl, heterocycloalkyl, alkylcycloalkyl, heteroalkylcycloalkyl, aryl, heteroaryl, aralkyl and heteroaralkyl, which groups are optionally substituted,

or a pharmaceutically acceptable salt, solvate or hydrate thereof.

The present invention relates to a compound of formula (II):

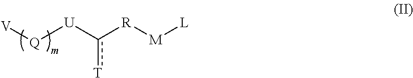

wherein

L is selected from OR³, C(O)R³, C(O)OR³, C(O)NR³R⁴, OP(O)(OR³)₂, C(O)OP(O)(OR³)₂, P(OR³)₃, C(O)OP(OR³)₃, C(O)P(OR³)₃, OS(O)(OR³)₂, C(O)S(O)(OR³)₂, OS(O)₂(OR³), C(O)S(O)₂(OR³), OSR³, C(O)SR³, OSR³R⁴, C(O)SR³R⁴, cycloalkyl, heterocycloalkyl and heteroaryl;

M is a hydrocarbon chain containing from 7 to 25 carbon atoms, wherein the hydrocarbon chain is unsaturated, branched or unbranched, and optionally includes one or more heteroatoms selected from O, N and S;

R and U are selected from CH₂, O and NR³, wherein R may form a 5- or 6-membered cycloalkyl or heterocycloalkyl ring with T and M;

T is selected from CH₂, O, NR³ and S;

Q is CH₂;

m is 0, 1 or 2;

V is selected from branched alkyl, heteroalkyl, alkenyl, alkynyl, cycloalkyl, heterocycloalkyl, aryl and heteroaryl, which groups are optionally substituted,

wherein R³ and R⁴ are independently selected from H, OH, alkyl, heteroalkyl, alkenyl, alkynyl, cycloalkyl, heterocycloalkyl, alkylcycloalkyl, heteroalkylcycloalkyl, aryl, heteroaryl, aralkyl and heteroaralkyl, which groups are optionally substituted,

or a pharmaceutically acceptable salt, solvate or hydrate thereof.

The invention also relates to compositions including the above described compounds, and to uses of the compounds and compositions for treating proliferative disease, for inducing apoptosis and/or for inhibiting proliferation or metastasis.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Graph showing the effect of compounds 12, 13, 14, 15 and 16 on caspase-3 activity in MDA-MB-231 cells, indicating cancer cell killing by apoptosis.

FIG. 2. Graph showing the effect of compounds 12, 13, 14, 15 and 16 of the present invention on Annexin V, indicating cancer cell killing by apoptosis.

FIG. 3. Graph showing the effect of compounds 12, 13, 14, 15 and 16 on migration of MDA-MB-231 cells out of matrigel droplets.

FIG. 4. Graph showing the effect of compound 15 on mouse body weight gain or loss.

FIG. 5. (a) Graph showing the effect of compound 15 on primary tumour growth in mice. (b) Graph showing the effect of compound 15 on primary tumour weight in mice.

FIG. 6. (a) Macroscopic appearance of tumour foci on mouse liver and spleen of control mice. (b) Macroscopic appearance of tumour foci on mouse liver and spleen of mice treated with compound 15.

FIG. 7. Graph showing the effect of compound 29 on mouse body weight gain or loss.

FIG. 8. Graph showing the effect of compound 29 on mouse body weight gain or loss.

FIG. 9. Graph showing the effect of compound 29 on primary tumour growth in mice (*P<0.05).

FIG. 10. Graph showing effects of compound 29 on JC-1 staining in MDA-MB-231 cells.

FIG. 11. Graph showing the relationship between the concentration of compound 29 and caspase-3/7 activity in MDA-MB-231 cells.

FIG. 12. Figure showing the decreased confluence of compound 29-treated MDA-MB-231 cells.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Compounds are generally described herein using standard nomenclature. For compounds having asymmetric centres, it will be understood that, unless otherwise specified, all of the optical isomers and mixtures thereof are encompassed. Compounds with two or more asymmetric elements can also be present as mixtures of diastereomers. In addition, compounds with carbon-carbon double bonds may occur in Z and E forms, with all isomeric forms of the compounds being included in the present invention unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope, i.e., an atom having the same atomic number but a different mass number. By way of general example, and without limitation, isotopes of hydrogen include tritium and deuterium and isotopes of carbon include ¹¹C, ¹³C, and ¹⁴C.

Compounds according to the formulae provided herein, which have one or more stereogenic centres, have an enantiomeric excess of at least 50%. For example, such compounds may have an enantiomeric excess of at least 60%, 70%, 80%, 85%, 90%, 95%, or 98%. Some embodiments of the compounds have an enantiomeric excess of at least 99%. It will be apparent that single enantiomers (optically active forms) can be obtained by asymmetric synthesis, synthesis from optically pure precursors, biosynthesis (for example, using modified CYP102 such as CYP BM-3) or by resolution of the racemates, for example, enzymatic resolution or resolution by conventional methods such as crystallization in the presence of a resolving agent, or chromatography, using, for example, a chiral HPLC column.

Certain compounds are described herein using a general formula that includes variables such as R¹, A, B, X, Y and Z. Unless otherwise specified, each variable within such a formula is defined independently of any other variable, and any variable that occurs more than one time in a formula is defined independently at each occurrence. Therefore, for example, if a group is shown to be substituted with 0, 1 or 2 R*, the group may be unsubstituted or substituted with up to two R* groups and R* at each occurrence is selected independently from the definition of R*. Also, combinations of substituents and/or variables are permissible only if such combinations result in stable compounds, i.e., compounds that can be isolated, characterized and tested for biological activity.

A “pharmaceutically acceptable salt” of a compound disclosed herein is an acid or base salt that is generally considered in the art to be suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids.

Suitable pharmaceutically acceptable salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzenesulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic (such as acetic, HOOC—(CH₂)ₙ—COOH where n is any integer from 0 to 6, i.e. 0, 1, 2, 3, 4, 5 or 6), and the like. Similarly, pharmaceutically acceptable cations include, but are not limited to sodium, potassium, calcium, aluminum, lithium and ammonium. A person skilled in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Generally, the use of nonaqueous media, such as ether, ethyl acetate, ethanol, isopropanol or acetonitrile, is preferred. It will be apparent that each compound of formula (I) and (II) may, but need not, be present as a hydrate, solvate or non-covalent complex. In addition, the various crystal forms and polymorphs are within the scope of the present invention, as are prodrugs of the compounds of formulae (I) and (II) provided herein.

A “prodrug” is a compound that may not fully satisfy the structural requirements of the compounds provided herein, but is modified in vivo, following administration to a subject or patient, to produce a compound of formula (I) or (II) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

A “substituent” as used herein, refers to a molecular moiety that is covalently bonded to an atom within a molecule of interest. For example, a “ring substituent” may be a moiety such as a halogen, alkyl group, heteroalkyl group, haloalkyl group or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e., a compound that can be isolated, characterized and tested for biological activity. When a substituent is oxo, i.e., ═O, then two hydrogens on the atom are replaced. An oxo group that is a substituent of an aromatic carbon atom results in a conversion of —CH— to —C(═O)— and a loss of aromaticity. For example a pyridyl group substituted by oxo is a pyridone. Examples of suitable substituents are alkyl (including haloalkyl e.g. CF₃), heteroalkyl, halogen (for example, fluorine, chlorine, bromine or iodine atoms), C(O)OR¹ (e.g. C(O)OH), C(O)OR³ (e.g. C(O)OH), C(O)R¹ (e.g. C(O)H), C(O)R³ (e.g. C(O)H), OH, ═O, SH, SO₃H, NH₂, NH-alkyl, NR¹₃⁺ (e.g. N(CH₃)₃⁺), NR³₃⁺ (e.g. N(CH₃)₃⁺), ═NH, N₃ and NO₂ groups.

The term “alkyl” refers to a saturated, straight-chain or branched hydrocarbon group that contains from 1 to 20 carbon atoms, preferably from 1 to 10 carbon atoms, for example a n-octyl group, especially from 1 to 6, i.e. 1, 2, 3, 4, 5, or 6, carbon atoms. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl.

The term “heteroalkyl” refers to an alkyl group as defined above that contains one or more heteroatoms selected from oxygen, nitrogen and sulphur (especially oxygen and nitrogen). Specific examples of heteroalkyl groups are methoxy, trifluoromethoxy, ethoxy, n-propyloxy, iso-propyloxy, butoxy, tert-butyloxy, methoxymethyl, ethoxymethyl, —CH₂CH₂OH, —CH₂OH, methoxyethyl, 1-methoxyethyl, 1-ethoxyethyl, 2-methoxyethyl or 2-ethoxyethyl, methylamino, ethylamino, propylamino, iso-propylamino, dimethylamino, diethylamino, iso-propyl-ethylamino, methylamino methyl, ethylamino methyl, di-iso-propylamino ethyl, methylthio, ethylthio, iso-propylthio, enol ether, dimethylamino methyl, dimethylamino ethyl, acetyl, propionyl, butyryloxy, acetyloxy, methoxycarbonyl, ethoxycarbonyl, propionyloxy, acetylamino, propionylamino, carboxymethyl, carboxyethyl, carboxypropyl, N-ethyl-N-methylcarbamoyl and N-methylcarbamoyl. Further examples of heteroalkyl groups are nitrile, iso-nitrile, cyanate, thiocyanate, isocyanate, iso-thiocyanate and alkylnitrile groups.

The term “alkenyl” refers to an at least partially unsaturated, straight-chain or branched hydrocarbon group that contains from 2 to 20 carbon atoms, preferably from 2 to 10 carbon atoms, especially from 2 to 6, i.e. 2, 3, 4, 5 or 6, carbon atoms. Specific examples of alkenyl groups are ethenyl (vinyl), propenyl (allyl), iso-propenyl, butenyl, ethinyl, propinyl, butinyl, acetylenyl, propargyl, iso-prenyl and hex-2-enyl group. Preferably, alkenyl groups have one or two double bond(s).

The term “alkynyl” refers to a at least partially unsaturated, straight-chain or branched hydrocarbon group that contains from 2 to 20 carbon atoms, preferably from 2 to 10 carbon atoms, especially from 2 to 6, i.e. 2, 3, 4, 5 or 6, carbon atoms. Specific examples of alkynyl groups are ethynyl, propynyl, butynyl, acetylenyl and propargyl groups. Preferably, alkynyl groups have one or two (especially preferably one) triple bond(s).

The term “cycloalkyl” refers to a saturated or partially unsaturated (for example, a cycloalkenyl group) cyclic group that contains one or more rings (preferably 1 or 2), and contains from 3 to 14 ring carbon atoms, preferably from 3 to 10 (especially 3, 4, 5, 6 or 7) ring carbon atoms. Specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, adamantane (i.e. tricycle[3.3.1.1^{3,7}]decane), cyclopentylcyclohexyl and cyclohex-2-enyl.

The term “heterocycloalkyl” refers to a cycloalkyl group as defined above in which one or more (preferably 1, 2 or 3) ring carbon atoms, each independently, have been replaced by an oxygen, nitrogen, silicon, selenium, phosphorus or sulfur atom (preferably by an oxygen, sulfur or nitrogen atom). A heterocycloalkyl group has preferably 1 or 2 rings containing from 3 to 10 (especially 3, 4, 5, 6 or 7) ring atoms (preferably selected from C, O, N and S). Specific examples are piperidyl, prolinyl, imidazolidinyl, piperazinyl, morpholinyl, urotropinyl, pyrrolidinyl, tetrahydrothiophenyl, tetrahydropyranyl, tetrahydrofuryl and 2-pyrazolinyl group and also lactames, lactones, cyclic imides and cyclic anhydrides.

The term “alkylcycloalkyl” refers to a group that contains both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two ring systems having from 3 to 10 (especially 3, 4, 5, 6 or 7) ring carbon atoms, and one alkyl, alkenyl or alkynyl group having 1 or 2 to 6 carbon atoms. The alkyl, alkenyl or alkynyl groups may form a bi- or tri-cyclic ring system with the cycloalkyl group, and may be the means by which the cycloalkyl group is joined to the compound of formula (I) or (II).

The term “heteroalkylcycloalkyl” refers to alkylcycloalkyl groups as defined above in which one or more, preferably 1, 2 or 3, carbon atoms have been replaced independently of each other by an oxygen, nitrogen, silicon, selenium, phosphorus or sulfur atom (preferably by an oxygen, sulfur or nitrogen atom). A heteroalkylcycloalkyl group preferably contains 1 or 2 ring systems having from 3 to 10 (especially 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkyl-heterocycloalkyl and heteroalkylheterocycloalkenyl, the cyclic groups being saturated or mono-, di- or tri-unsaturated.

The term “aryl” refers to an aromatic group that contains one or more rings containing from 6 to 14 ring carbon atoms, preferably from 6 to 10 (especially 6) ring carbon atoms. Examples are phenyl, naphthyl and biphenyl groups.

The term “heteroaryl” refers to an aromatic group that contains one or more rings containing from 5 to 14 ring atoms, preferably from 5 to 10 (especially 5 or 6) ring atoms, and contains one or more (preferably 1, 2, 3 or 4) oxygen, nitrogen, phosphorus or sulfur ring atoms (preferably O, S or N). Examples are pyridyl (for example, 4-pyridyl), imidazolyl (for example, 2-imidazolyl), phenylpyrrolyl (for example, 3-phenylpyrrolyl), thiazolyl, iso-thiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (for example, 3-pyrazolyl) and iso-quinolinyl groups.

The term “aralkyl” refers to a group containing both aryl and also alkyl, alkenyl, alkynyl and/or cycloalkyl groups in accordance with the above definitions, such as, for example, an arylalkyl, arylalkenyl, arylalkynyl, arylcycloalkyl, aryl-cycloalkenyl, alkylarylcycloalkyl and alkylarylcycloalkenyl group. The alkyl, alkenyl or alkynyl groups may provide the means by which the alkyl group is joined to the compound of formula (I) or (II). Specific examples of aralkyls are 1H-indene, tetraline, dihydronaphthalene, indanone, phenylcyclopentyl, cyclohexylphenyl, fluorene and indane. An aralkyl group preferably contains one or two aromatic ring systems (1 or 2 rings) containing from 6 to 10 carbon atoms and one alkyl, alkenyl and/or alkynyl group containing from 1 or 2 to 6 carbon atoms and/or a cycloalkyl group containing 5 or 6 ring carbon atoms.

The term “heteroaralkyl” refers to an aralkyl group as defined above in which one or more (preferably 1, 2, 3 or 4) carbon atoms, each independently, have been replaced by an oxygen, nitrogen, silicon, selenium, phosphorus, boron or sulfur atom (preferably oxygen, sulfur or nitrogen). That is, a group containing aryl or heteroaryl, respectively, and also alkyl, alkenyl, alkynyl and/or heteroalkyl and/or cycloalkyl and/or heterocycloalkyl groups in accordance with the above definitions. A heteroaralkyl group preferably contains one or two aromatic ring systems (1 or 2 rings) containing from 5 or 6 to 10 ring carbon atoms and one alkyl, alkenyl and/or alkynyl group containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms. The alkyl, alkenyl or alkynyl group may provide the means by which the alkyl group is joined to the compound of formula (I) or (II).

Examples are arylheteroalkyl, arylheterocycloalkyl, arylheterocycloalkenyl, arylalkylheterocycloalkyl, arylalkenyl-heterocycloalkyl, arylalkynylheterocycloalkyl, arylalkyl-heterocycloalkenyl, heteroarylalkyl, heteroarylalkenyl, heteroarylalkynyl, heteroarylheteroalkyl, heteroaryl-cycloalkyl, heteroarylcycloalkenyl, heteroarylhetero-cycloalkyl, heteroarylheterocycloalkenyl, heteroarylalkyl-cycloalkyl, heteroarylalkylheterocycloalkenyl, heteroaryl-heteroalkylcycloalkyl, heteroarylheteroalkylcycloalkenyl and heteroarylheteroalkylheterocycloalkyl groups, the cyclic groups being saturated or mono-, di- or tri-unsaturated. Specific examples are tetrahydroisoquinolinyl and benzoyl.

The expression “halogen” or “halogen atom” as used herein means fluorine, chlorine, bromine, or iodine.

The term “optionally substituted” refers to a group in which one, two, three or more hydrogen atoms have been replaced independently of each other by halogen (for example, fluorine, chlorine, bromine or iodine atoms) and/or by C(O)OR¹ (e.g. C(O)OH), C(O)OR³ (e.g. C(O)OH), C(O)R¹ (e.g. C(O)H), C(O)R³ (e.g. C(O)H), OH, ═O, SH, ═S, SO₃H, NH₂, NH-alkyl, NR¹₃⁺ (e.g. N(CH₃)₃⁺), NR³₃⁺ (e.g. N(CH₃)₃⁺), ═NH, N₃ or NO₂ groups. This expression also refers to a group that is substituted by one, two, three or more alkyl, alkenyl, alkynyl, heteroalkyl, cycloalkyl, heterocycloalkyl, alkylcycloalkyl, heteroalkylcycloalkyl, aryl, heteroaryl, aralkyl or heteroaralkyl groups. These groups may themselves be substituted. For example, an alkyl group substituent may be substituted by one or more halogen atoms (i.e. may be a haloalkyl group). The term “haloalkyl” refers to an alkyl group (as defined above) that is substituted by one or more halogen atoms (as also defined above). Specific examples of haloalkyl groups are trifluoromethyl, dichloroethyl, dichloromethyl and iodoethyl.

As used herein a wording defining the limits of a range of length such as, for example, “from 1 to 5” means any integer from 1 to 5, i. e. 1, 2, 3, 4 and 5. In other words, any range defined by two integers explicitly mentioned is meant to comprise and disclose any integer defining said limits and any integer comprised in said range.

Preferred compounds of formula (I) are those where Z is a cycloalkyl group, an aryl group or a branched alkyl group (for example, a tert-butyl group). Preferably, the cycloalkyl group is a cyclohexyl group and the aryl group is a phenyl group. In the embodiment where Z is an aryl group (e.g. a phenyl group), the aryl group may be substituted by either a halogen (for example, fluorine, chlorine or iodine) or an alkyl group (for example, methyl).

The aryl group (e.g. phenyl group) may also be substituted by one or more halogens, one or more alkyl groups, one or more heteroalkyl groups, or combinations thereof. In one embodiment, the aryl group is substituted by a heteroalkyl group (e.g. a methoxy group). In, another embodiment, the aryl group is substituted by two halogens. The aryl group may be substituted by a halogen and an alkyl group, and the alkyl group may be a substituted alkyl group (e.g. substituted by two or more halogen atoms). In one embodiment, the substituted alkyl group is CF₃.

In one embodiment, the compound of formula (I) is a compound of formula (Ia):

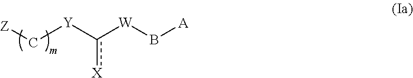

wherein

A is selected from OR¹, C(O)R¹, C(O)OR¹, C(O)NR¹R², OP(O)(OR¹)₂, C(O)OP(O)(OR¹)₂, P(OR¹)₃, C(O)OP(OR¹)₃, C(O)P(OR¹)₃, OS(O)(OR¹)₂, C(O)S(O)(OR¹)₂, OS(O)₂(OR¹), C(O)S(O)₂(OR¹), OSR¹, C(O)SR¹, OSR¹R², C(O)SR¹R², cycloalkyl, heterocycloalkyl and heteroaryl;

B is a hydrocarbon chain containing from 7 to 25 carbon atoms, wherein the hydrocarbon chain is saturated, branched or unbranched, and optionally includes one or more heteroatoms selected from O, N and S;

W and Y are selected from CH₂, O and NR¹, wherein W may form a 5- or 6-membered cycloalkyl or heterocycloalkyl ring with X and B;

X is selected from CH₂, O, NR¹ and S;

C is CH₂;

m is 0, 1 or 2;

Z is selected from cycloalkyl, heterocycloalkyl, aryl and heteroaryl, which groups are optionally substituted,

wherein R¹ and R² are independently selected from H, OH, alkyl, heteroalkyl, alkenyl, alkynyl, cycloalkyl, heterocycloalkyl, alkylcycloalkyl, heteroalkylcycloalkyl, aryl, heteroaryl, aralkyl and heteroaralkyl, which groups are optionally substituted,

or a pharmaceutically acceptable salt, solvate or hydrate thereof.

In one embodiment, Z is an aryl or heteroaryl group. Preferably, Z is an aryl group. The aryl group may be a phenyl group.

Z may be substituted by one or more halogens, one or more alkyl groups, one or more heteroalkyl groups, or combinations thereof. In one embodiment, Z is substituted by an electron-withdrawing group (e.g. CN, C(O)OR¹ (e.g. C(O)OH), C(O)OR³ (e.g. C(O)OH), C(O)R¹ (e.g. C(O)H), C(O)R³ (e.g. C(O)H), CCl₃, NO₂, CF₃, SO₃H, NR¹₃⁺ (e.g. N(CH₃)₃⁺), NR³₃⁺ (e.g. N(CH₃)₃⁺)). Z may be substituted by two halogens. Z may be substituted by a halogen and an alkyl group, and the alkyl group may be a substituted alkyl group (e.g. substituted by two or more halogen atoms). In one embodiment, the substituted alkyl group is CF₃. Z may also be substituted by a heteroalkyl group (e.g. a methoxy group).

Preferred compounds are also those where the hydrocarbon chain contains from 7 to 25 carbon atoms (for example, between 10 and 21 carbon atoms). Accordingly, the hydrocarbon chain may contain 7, 8, 9, 10, 11, 12, 13, 14, 15, 16, 17, 18, 19, 20, 21, 22, 23, 24 or 25 carbon atoms.

Preferred compounds also include those where W and Y are both NH, X is O and the bond between X and the carbon to which X is attached is a double bond.

Preferred compounds of formula (II) are those where V is a cycloalkyl group, an aryl group or a branched alkyl group (for example, a tert-butyl group). Preferably, the cycloalkyl group is a cyclohexyl group and the aryl group is a phenyl group. In the embodiment where V is an aryl group, the aryl group may be substituted by either a halogen (for example, fluorine, chlorine or iodine) or an alkyl group (for example, methyl).

The aryl group (e.g. phenyl group) may also be substituted by one or more halogens, one or more alkyl groups, one or more heteroalkyl groups, or combinations thereof. In one embodiment, the aryl group is substituted by a heteroalkyl group (e.g. a methoxy group). In another embodiment, the aryl group is substituted by two halogens. The aryl group may be substituted by a halogen and an alkyl group, and the alkyl group may be a substituted alkyl group (e.g. substituted by two or more halogen atoms). In one embodiment, the substituted alkyl group is CF₃.

In one embodiment, the compound of formula (II) is a compound of formula (IIa):

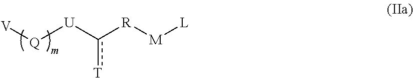

wherein

L is selected from OR³, C(O)R³, C(O)OR³, C(O)NR³R⁴, OP(O)(OR³)₂, C(O)OP(O)(OR³)₂, P(OR³)₃, C(O)OP(OR³)₃, C(O)P(OR³)₃, OS(O)(OR³)₂, C(O)S(O)(OR³)₂, OS(O)₂(OR³), C(O)S(O)₂(OR³), OSR³, C(O)SR³, OSR³R⁴, C(O)SR³R⁴, cycloalkyl, heterocycloalkyl and heteroaryl;

M is a hydrocarbon chain containing from 7 to 25 carbon atoms, wherein the hydrocarbon chain is unsaturated, branched or unbranched, and optionally includes one or more heteroatoms selected from O, N and S;

R and U are selected from CH₂, O and NR³, wherein R may form a 5- or 6-membered cycloalkyl or heterocycloalkyl ring with T and M;

T is selected from CH₂, O, NR³ and S;

Q is CH₂;

m is 0, 1 or 2;

V is selected from cycloalkyl, heterocycloalkyl, aryl and heteroaryl, which groups are optionally substituted,

wherein R³ and R⁴ are independently selected from H, OH, alkyl, heteroalkyl, alkenyl, alkynyl, cycloalkyl, heterocycloalkyl, alkylcycloalkyl, heteroalkylcycloalkyl, aryl, heteroaryl, aralkyl and heteroaralkyl, which groups are optionally substituted,

or a pharmaceutically acceptable salt, solvate or hydrate thereof.

In one embodiment, V is an aryl or heteroaryl group. Preferably, V is an aryl group. The aryl group may be a phenyl group.

V may be substituted by one or more halogens, one or more alkyl groups, one or more heteroalkyl groups, or combinations thereof. In one embodiment, V is substituted by an electron-withdrawing group (e.g. CN, C(O)OR¹ (e.g. C(O)OH), C(O)OR³ (e.g. C(O)OH), C(O)R¹ (e.g. C(O)H), C(O)R³ (e.g. C(O)H), CCl₃, NO₂, CF₃, SO₃H, NR¹₃⁺ (e.g. N(CH₃)₃⁺), NR³₃⁺ (e.g. N(CH₃)₃⁺)). V may be substituted by two halogens. V may be substituted by a halogen and an alkyl group, and the alkyl group may be a substituted alkyl group (e.g. substituted by two or more halogen atoms). In one embodiment, the substituted alkyl group is CF₃. V may also be substituted by a heteroalkyl group (e.g. a methoxy group).

Preferred compounds of formula (II) are also those where the hydrocarbon chain contains from 7 to 25 carbon atoms (for example, between 10 and 21 carbon atoms). Accordingly, the hydrocarbon chain may contain 7, 8, 9, 10, 11, 12, 13, 14, 15, 16, 17, 18, 19, 20, 21, 22, 23, 24 or 25 carbon atoms.

Preferred compounds of formula (II) include those where Q includes one or more Z double bonds. Q may also include a mixture of E and Z double bonds, or just E double bonds.

Preferred compounds also include those where R and U are both NH, T is O and the bond between T and the carbon to which T is attached is a double bond.

Specific examples of the compounds of the present invention are given in Table 1, below.

TABLE 1

| Compound | Structure |
|---|---|
| 1: EUCy | 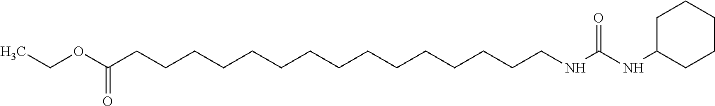 [SMILES: CCOC(=O)CCCCCCCCCCCCCCCNC(=O)NC1CCCCC1] |
| 2: EUEPh | 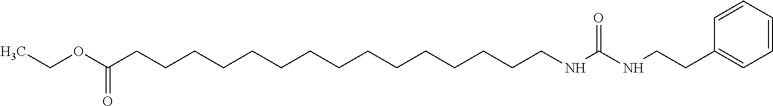 [SMILES: CCOC(=O)CCCCCCCCCCCCCCCNC(=O)NCCc1ccccc1] |
| 3: CUEPh | 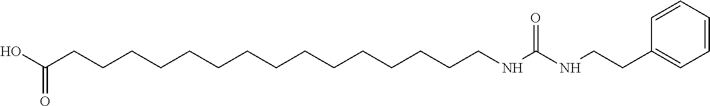 [SMILES: O=C(O)CCCCCCCCCCCCCCCNC(=O)NCCc1ccccc1] |
| 4: EUtB | 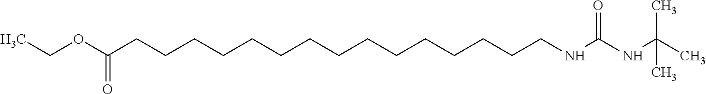 [SMILES: CCOC(=O)CCCCCCCCCCCCCCCNC(=O)NC(C)(C)C] |
| 5: CUCy | 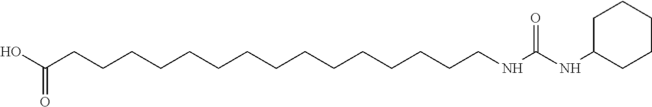 [SMILES: O=C(O)CCCCCCCCCCCCCCCNC(=O)NC1CCCCC1] |
| 6: CUPh | 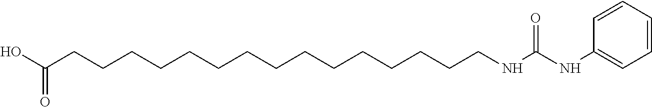 [SMILES: O=C(O)CCCCCCCCCCCCCCCNC(=O)Nc1ccccc1] |
| 7: CUtB | 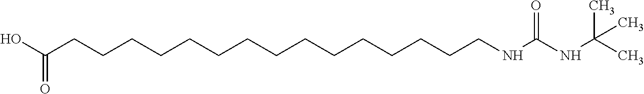 [SMILES: CC(C)(C)NC(=O)NCCCCCCCCCCCCCCCC(=O)O] |
| 8: CUBz | 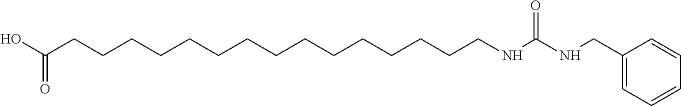 [SMILES: O=C(O)CCCCCCCCCCCCCCCNC(=O)NCc1ccccc1] |
| 9: EUPh | 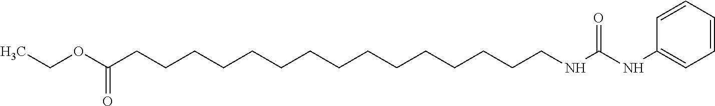 [SMILES: CCOC(=O)CCCCCCCCCCCCCCCNC(=O)Nc1ccccc1] |
| 10: EUBz | 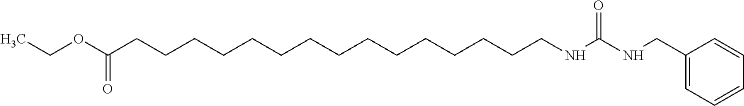 [SMILES: CCOC(=O)CCCCCCCCCCCCCCCNC(=O)NCc1ccccc1] |
| 11: EUPhF | 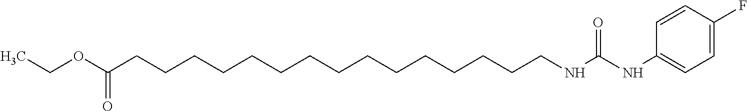 [SMILES: CCOC(=O)CCCCCCCCCCCCCCCNC(=O)Nc1ccc(F)cc1] |
| 12: CUPhF | 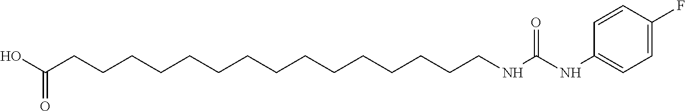 [SMILES: O=C(O)CCCCCCCCCCCCCCCNC(=O)Nc1ccc(F)cc1] |
| 13: EUPhCl | 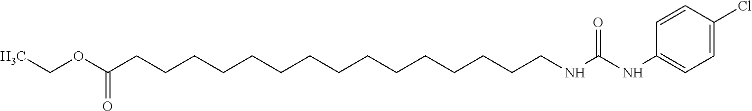 [SMILES: CCOC(=O)CCCCCCCCCCCCCCCNC(=O)Nc1ccc(Cl)cc1] |
| 14: CUPhCl | 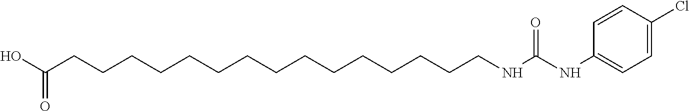 [SMILES: O=C(O)CCCCCCCCCCCCCCCNC(=O)Nc1ccc(Cl)cc1] |
| 15: EUT | 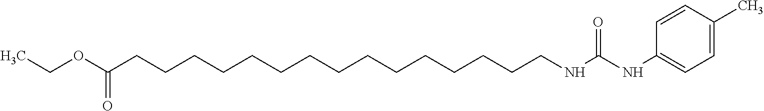 [SMILES: CCOC(=O)CCCCCCCCCCCCCCCNC(=O)Nc1ccc(C)cc1] |
| 16: CUT | 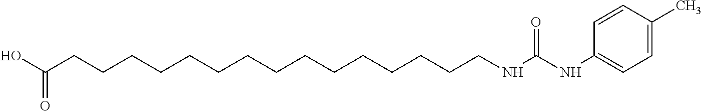 [SMILES: Cc1ccc(NC(=O)NCCCCCCCCCCCCCCCC(=O)O)cc1] |
| 17: EUA | 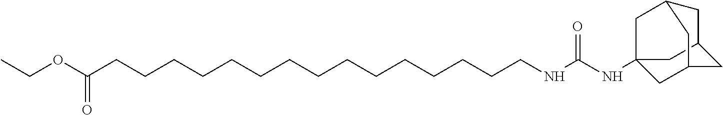 [SMILES: CCOC(=O)CCCCCCCCCCCCCCCNC(=O)NC12CC3CC(CC(C3)C1)C2] |
| 18: CUA | 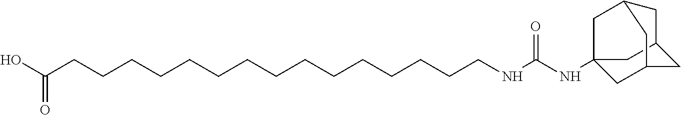 [SMILES: O=C(O)CCCCCCCCCCCCCCCNC(=O)NC12CC3CC(CC(C3)C1)C2] |
| 19: CUE | 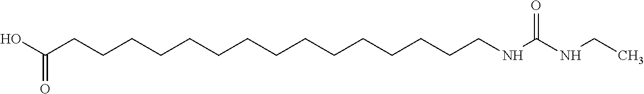 [SMILES: CCNC(=O)NCCCCCCCCCCCCCCCC(=O)O] |
| 20: EUE | 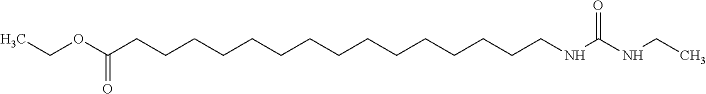 [SMILES: CCNC(=O)NCCCCCCCCCCCCCCCC(=O)OCC] |
| 21 | 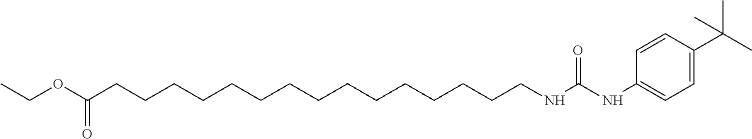 [SMILES: CCOC(=O)CCCCCCCCCCCCCCCNC(=O)Nc1ccc(C(C)(C)C)cc1] |
| 22 | 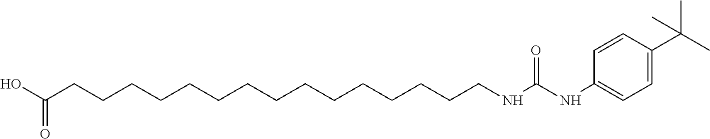 [SMILES: CC(C)(C)c1ccc(NC(=O)NCCCCCCCCCCCCCCCC(=O)O)cc1] |
| 23 | 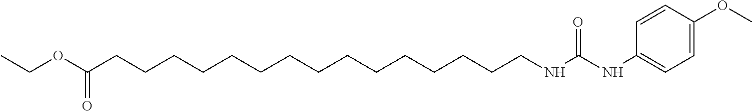 [SMILES: CCOC(=O)CCCCCCCCCCCCCCCNC(=O)Nc1ccc(OC)cc1] |
| 24 | 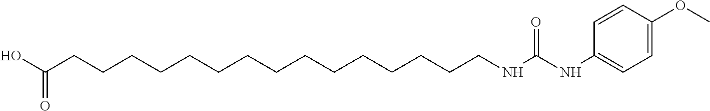 [SMILES: COc1ccc(NC(=O)NCCCCCCCCCCCCCCCC(=O)O)cc1] |
| 25 | 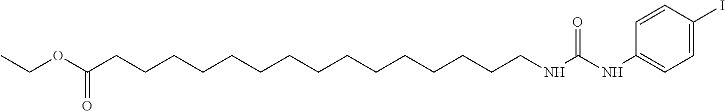 [SMILES: CCOC(=O)CCCCCCCCCCCCCCCNC(=O)Nc1ccc(I)cc1] |
| 26 | 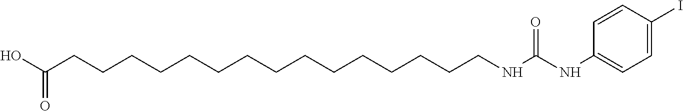 [SMILES: O=C(O)CCCCCCCCCCCCCCCNC(=O)Nc1ccc(I)cc1] |
| 27 | 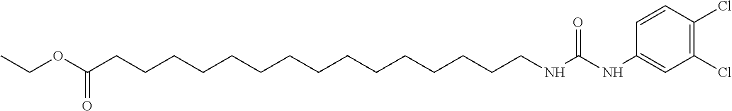 [SMILES: CCOC(=O)CCCCCCCCCCCCCCCNC(=O)Nc1ccc(Cl)c(Cl)c1] |
| 28 | 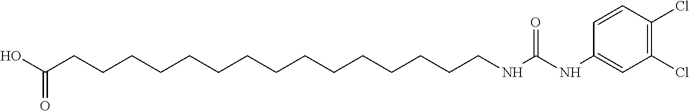 [SMILES: O=C(O)CCCCCCCCCCCCCCCNC(=O)Nc1ccc(Cl)c(Cl)c1] |
| 29 | 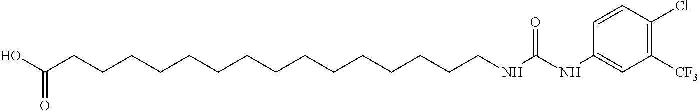 [SMILES: O=C(O)CCCCCCCCCCCCCCCNC(=O)Nc1ccc(Cl)c(C(F)(F)F)c1] |
| 30 | 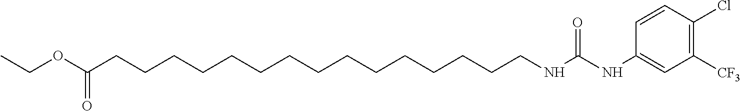 [SMILES: CCOC(=O)CCCCCCCCCCCCCCCNC(=O)Nc1ccc(Cl)c(C(F)(F)F)c1] |
| 31 | 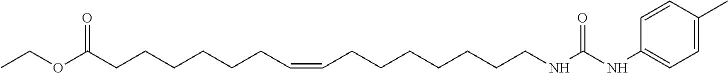 [SMILES: CCOC(=O)CCCCCC/C=C\CCCCCCCNC(=O)Nc1ccc(C)cc1] |

In one embodiment, the compound of formula (I) is selected from the group consisting of compounds 6, 12, 13, 14, 15, 16, 19, 24, 26, 28 and 29 from Table 1 above.

In another embodiment, the compound of formula (II) is selected from the group consisting of compound 31 from Table 1 above.

The compounds of the present invention can be synthesised by any suitable method known to a person skilled in the art. A general synthesis is given below in Schemes 1(a), 1(b) and 1(c).

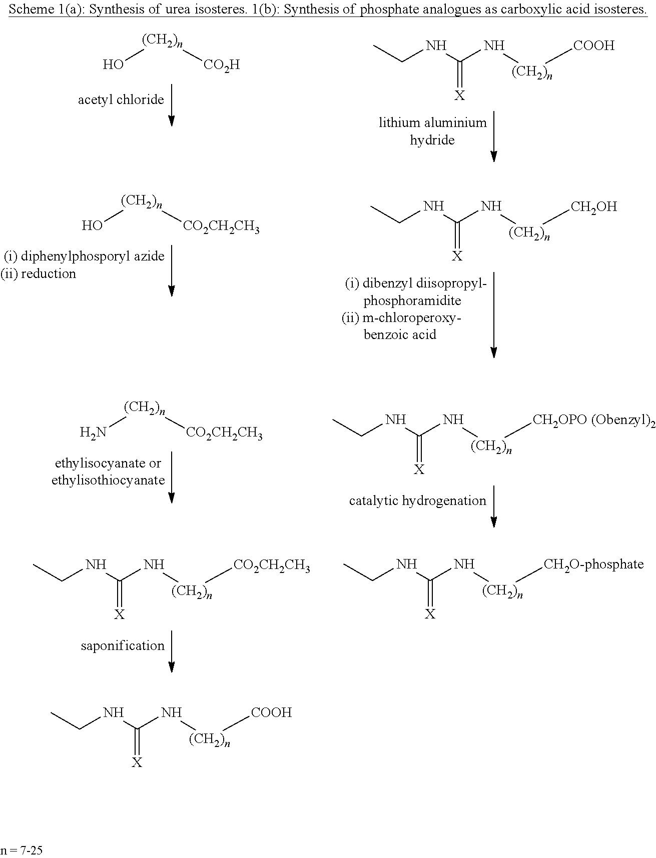

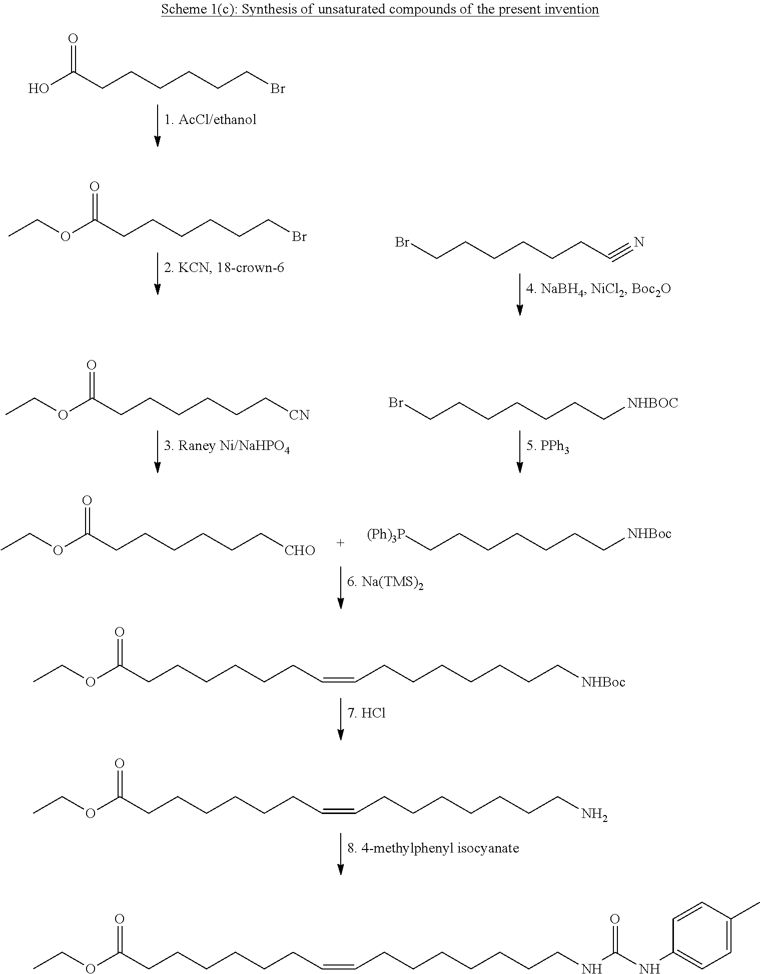

A person skilled in the art will understand that if analogues bearing, for example, branched alkyl, aryl or cycloalkyl groups are desired, the corresponding starting materials (for example, cycloalkyl- or aryl-isocyanates, or branched alkyl-isocyanates) will need to be used.

The compounds of the present invention may exhibit high anti-proliferative activity and in particular, high efficacy against metastatic disorders. Specifically, in the examples herein, specific compounds are shown to inhibit proliferation, to induce markers of apoptosis and/or to inhibit cell migration. The present inventors have found that compounds containing an aryl group (e.g. where Z or V is a phenyl group), and where the aryl group is further substituted by one or more electron-withdrawing groups (such as NO₂, CF₃ and/or SO₃H), are particularly effective at inducing apoptosis in primary cancer cells.

Cells undergoing proliferation may be generally classified as cells in the G₁, S, G₂ or M phase of the cell cycle. In certain embodiments, a compound of the invention may inhibit a cell from entering or from leaving any one of these phases, for example by inducing apoptosis or cell death.

I certain embodiments, the compounds of the present invention may be resistant to cEH-dependent hydration, but still have the beneficial anti-proliferative activity of omega-3 17,18-epoxy-EPA.

The therapeutic use of compounds of formulae (I) and (II), their pharmaceutically acceptable salts, solvates or hydrates and also formulations and pharmaceutical compositions (including mixtures of the compounds of formulae (I) and/or (II)) are within the scope of the present invention. Accordingly, the present invention also relates to pharmaceutical compositions including a therapeutically effective amount of the compounds of formula (I), or its pharmaceutically acceptable salt, solvate or hydrate thereof, and one or more pharmaceutically acceptable excipients. The present invention also relates to pharmaceutical compositions including a therapeutically effective amount of the compounds of formula (II), or its pharmaceutically acceptable salt, solvate or hydrate thereof, and one or more pharmaceutically acceptable excipients.

The pharmaceutical compositions according to the present invention include at least one compound of formula (I) and/or (II) and, optionally, one or more carrier substances, for example, cyclodextrins such as hydroxypropyl β-cyclodextrin, micelles or liposomes, excipients and/or adjuvants. Pharmaceutical compositions may additionally include, for example, one or more of water, buffers (for example, neutral buffered saline or phosphate buffered saline), ethanol, mineral oil, vegetable oil, dimethylsulfoxide, carbohydrates (for example, glucose, mannose, sucrose and mannitol), proteins, adjuvants, polypeptides or amino acids such as glycine, antioxidants, chelating agents such as EDTA or glutathione, and/or preservatives.

Further, one or more other active ingredients may, but need not, be included in the pharmaceutical compositions provided herein. For instance, the compounds of the invention may advantageously be employed in combination with an antibiotic, antifungal, or antiviral agent, antihistamine, a non-steroidal anti-inflammatory drug, a disease modifying antirheumatic drug, a cytostatic drug, a drug with smooth muscle modulatory activity, an inhibitor of one or more of the enzymes that process the compounds of the present invention and lead to a decrease in their efficacy (for example, a cEH inhibitor), or mixtures of these.

Pharmaceutical compositions may be formulated for any appropriate route of administration including, for example, topical (for example, transdermal or ocular), oral, buccal, nasal, vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. Within yet other embodiments, compositions provided herein may be formulated as a lyophilizate. Formulation for topical administration may be preferred for certain conditions such as in the treatment of skin conditions (for example, burns, itches or skin cancers).

Particularly preferred formulations for parenteral administration are liposomal formulations of the active compound (i.e. where the active compounds are contained or encapsulated in liposomes).

Liposomes comprising the compounds of the invention can be made by standard techniques including forming an organic solution having one or more compounds of the invention dissolved therein, contacting the organic solution with an aqueous solution and providing conditions for formation of a liposome therefrom.

A liposome may have a pH sensitivity of about pH 7.0, which means that the liposome is unstable below pH 7.0 such that the lipid bilayer of the liposome is disrupted below pH 7.0. A liposome may have a diameter ranging between about 50 nm and 200 μm. Accordingly, the liposome may be a small, sonicated unilamellar vesicle (SUV), a large unilamellar vesicle (LUV), or a liposome prepared by reverse phase evaporation (a REV), by french press (a FPV) or by ether injection (an EIV). Methods of preparing liposomes of such sizes, including methods of fractionating and purifying liposomes of the desired size, are known to a person skilled in the art.

A liposome may be unilamellar with respect to the liposome lipid bilayer. However, it will be understood that the liposome may comprise more than one lipid bilayer. Therefore, in one embodiment, the liposome may be a multilamellar vesicle such as a large, vortexed multilamellar vesicle (MLV).

A compound for providing the liposome with a charge for binding the liposome to a target cell may be advantageous for improving the fusion between the target lipid bilayer and the liposome bilayer. For example, DOTAP is particularly useful as a binding means for binding the liposome lipid bilayer to a target cell.

In one embodiment, a compound of the invention may be comprised in a layer of the lipid bilayer of the liposome. In this embodiment, a less hydrophobic portion of the molecule may be in contact with an inner aqueous core of the liposome, or in contact with an aqueous solution in which the liposome is contained.

Where the compound of the invention is provided in the form of a liposome as discussed above, in one embodiment the compound is administered to an individual requiring treatment by administration of a liposome including the compound, or a composition including said liposome, to an individual by injection.

Compositions intended for oral use may further comprise one or more components such as sweetening agents, flavoring agents, coloring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatin or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent such as calcium carbonate, calcium phosphate or kaolin, or as soft gelatin capsules wherein the active ingredient is mixed with water or an oil medium such as peanut oil, liquid paraffin or olive oil.

Aqueous suspensions contain the active ingredient(s) in admixture with excipients suitable for the manufacture of aqueous suspensions. Such excipients include suspending agents such as sodium carboxymethylcellulose, methylcellulose, hydropropylmethylcellulose, sodium alginate, polyvinylpyrrolidone, gum tragacanth and gum acacia, and dispersing or wetting agents such as naturally-occurring phosphatides (for example, lecithin), condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol mono-oleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and coloring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

Compounds may be formulated for local or topical administration, such as for topical application to the skin or mucous membranes, such as in the eye. Formulations for topical administration typically comprise a topical vehicle combined with active agent(s), with or without additional optional components. Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerin), glycols such as butylene, isoprene or propylene glycol, aliphatic alcohols such as lanolin, mixtures of water and organic solvents and mixtures of organic solvents such as alcohol and glycerin, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatin-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturizers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminum silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquatemiums, hydroxyethylceilulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerin, propylene glycol, and butylene glycol. Suitable emollients include lanolin alcohol, lanolin, lanolin derivatives, cholesterol, petrolatum, isostearyl neopentanoate and mineral oils. Suitable fragrances and colors include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch).

A pharmaceutical composition may be formulated as inhaled formulations, including sprays, mists, or aerosols. For inhalation formulations, the compounds provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient. Pharmaceutical compositions may also be prepared in the form of suppositories such as for rectal administration. Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations such as a capsule that creates a slow release of modulator following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of modulator release. The amount of modulator contained within a sustained release formulation depends upon, for example, the site of implantation, the rate and expected duration of release and the nature of the condition to be treated or prevented.

For the treatment of proliferative disorders, especially metastatic disorders, the dose of the biologically active compound according to the invention may vary within wide limits and may be adjusted to individual requirements. Active compounds according to the present invention are generally administered in a therapeutically effective amount. Preferred doses range from about 0.1 mg to about 140 mg per kilogram of body weight per day (e.g. about 0.5 mg to about 7 g per patient per day). The daily dose may be administered as a single dose or in a plurality of doses. The amount of active ingredient that may be combined with the carrier materials to produce a single dosage form will vary depending upon the host treated and the particular mode of administration. Dosage unit forms will generally contain between about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient), and the severity of the particular disorder undergoing therapy.

The terms “therapeutically effective amount” or “effective amount” refer to an amount of the compound of formula (I) that results in an improvement or remediation of the symptoms of a proliferative and/or metastatic disorder. The terms “therapeutically effective amount” or “effective amount” also refer to an amount of the compound of formula (II) that results in an improvement or remediation of the symptoms of a proliferative and/or metastatic disorder.

Preferred compounds of the invention will have certain pharmacological properties. Such properties include, but are not limited to oral bioavailability, such that the preferred oral dosage forms discussed above can provide therapeutically effective levels of the compound in vivo.

The compounds of the present invention are preferably administered to a patient (for example, a human) orally or parenterally, and are present within at least one body fluid or tissue of the patient. Accordingly, the present invention further provides methods for treating patients suffering from proliferative disorders (including metastatic disorders). As used herein, the term “treatment” encompasses both disorder-modifying treatment and symptomatic treatment, either of which may be prophylactic, i.e. before the onset of symptoms, in order to prevent, delay or reduce the severity of symptoms, or therapeutic, i.e. after the onset of symptoms, in order to reduce the severity and/or duration of symptoms. Patients may include but are not limited to primates, especially humans, domesticated companion animals such as dogs, cats, horses, and livestock such as cattle, pigs, sheep, with dosages as described herein.

Compounds of the present invention may be useful for the treatment and/or prevention of conditions and disorders associated with cell proliferation (including metastasis). Accordingly, the present invention also relates to a method of treating or preventing a proliferative disorder in a patient including administration to the patient of a therapeutically effective amount of a compound of formula (I), or a pharmaceutically-acceptable salt, solvate or hydrate thereof. The present invention also relates to the use of a therapeutically effective amount of a compound of formula (I), or a pharmaceutically-acceptable salt, solvate or hydrate thereof, for treating or preventing a proliferative disorder. The present invention also provides a pharmaceutical composition for use in treating or preventing a proliferative disorder, in any of the embodiments described in the specification. The present invention also relates to the use of a therapeutically effective amount of a compound of formula (I), or a pharmaceutically acceptable salt, solvate or hydrate thereof, for the manufacture of a medicament for treating or preventing a proliferative disorder.

The present invention also relates to a compound of formula (I), or a pharmaceutically acceptable salt, solvate or hydrate thereof, when used in a method of treating or preventing a proliferative disorder. The present invention also relates to a composition having an active ingredient for use in treating or preventing a proliferative disorder, wherein the active ingredient is a compound of formula (I), or a pharmaceutically acceptable salt, solvate or hydrate thereof. The present invention also relates to the use of a pharmaceutical composition containing a compound of the formula (I), or a pharmaceutically acceptable salt, solvate or hydrate thereof, in treating or preventing a proliferative disorder, such as described above. In one embodiment, the compound of formula (I) is essentially the only active ingredient of the composition. In one embodiment, the proliferative disorder is a metastatic disorder.

The present invention also relates to a method of treating or preventing a proliferative disorder in a patient including administration to the patient of a therapeutically effective amount of a compound of formula (II), or a pharmaceutically-acceptable salt, solvate or hydrate thereof. The present invention also relates to the use of a therapeutically effective amount of a compound of formula (II), or a pharmaceutically-acceptable salt, solvate or hydrate thereof, for treating or preventing a proliferative disorder. The present invention also provides a pharmaceutical composition for use in treating or preventing a proliferative disorder, in any of the embodiments described in the specification. The present invention also relates to the use of a therapeutically effective amount of a compound of formula (II), or a pharmaceutically acceptable salt, solvate or hydrate thereof, for the manufacture of a medicament for treating or preventing a proliferative disorder.

The present invention also relates to a compound of formula (II), or a pharmaceutically acceptable salt, solvate or hydrate thereof, when used in a method of treating or preventing a proliferative disorder. The present invention also relates to a composition having an active ingredient for use in treating or preventing a proliferative disorder, wherein the active ingredient is a compound of formula (II), or a pharmaceutically acceptable salt, solvate or hydrate thereof. The present invention also relates to the use of a pharmaceutical composition containing a compound of the formula (II), or a pharmaceutically acceptable salt, solvate or hydrate thereof, in treating or preventing a proliferative disorder, such as described above. In one embodiment, the compound of formula (II) is essentially the only active ingredient of the composition. In one embodiment, the proliferative disorder is a metastatic disorder.

Examples of conditions and disorders associated with cell proliferation include tumors or neoplasms, where proliferation of cells is uncontrolled and progressive. Some such uncontrolled proliferating cells are benign, but others are termed “malignant” and may lead to death of the organism. Malignant neoplasms or “cancers” are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and greater loss of their organization relative to one another and their surrounding tissues. This property is also called “anaplasia”. Neoplasms treatable by the present invention also include solid phase tumors/malignancies, i. e. carcinomas, locally advanced tumors and human soft tissue sarcomas. Carcinomas include those malignant neoplasms derived from epithelial cells that infiltrate (invade) the surrounding tissues and give rise to metastastic cancers, including lymphatic metastases. The compounds of the present invention have been found to be particularly effective against metastatic cancers (including in models of cell proliferation, migration, invasion and angiogenesis). The compounds of the present invention have also been found to be particularly effective at killing primary cancer cells.

Adenocarcinomas are carcinomas derived from glandular tissue, or which form recognizable glandular structures. Another broad category of cancers includes sarcomas, which are tumors whose cells are embedded in a fibrillar or homogeneous substance like embryonic connective tissue.

The invention also enables treatment of cancers of the myeloid or lymphoid systems, including leukemias, lymphomas and other cancers that typically do not present as a tumor mass, but are distributed in the vascular or lymphoreticular systems.

The type of cancer or tumor cells that may be amenable to treatment according to the invention include, for example, breast, colon, lung, and prostate cancers, gastrointestinal cancers including esophageal cancer, stomach cancer, colorectal cancer, polyps associated with colorectal neoplasms, pancreatic cancer and gallbladder cancer, cancer of the adrenal cortex, ACTH-producing tumor, bladder cancer, brain cancer including intrinsic brain tumors, neuroblastomas, astrocytic brain tumors, gliomas, and metastatic tumor cell invasion of the central nervous system, Ewing's sarcoma, head and neck cancer including mouth cancer and larynx cancer, kidney cancer including renal cell carcinoma, liver cancer, lung cancer including small and non-small cell lung cancers, malignant peritoneal effusion, malignant pleural effusion, skin cancers including malignant melanoma, tumor progression of human skin keratinocytes, squamous cell carcinoma, basal cell carcinoma, and hemangiopericytoma, mesothelioma, Kaposi's sarcoma, bone cancer including osteomas and sarcomas such as fibrosarcoma and osteosarcoma, cancers of the female reproductive tract including uterine cancer, endometrial cancer, ovarian cancer, ovarian (germ cell) cancer and solid tumors in the ovarian follicle, vaginal cancer, cancer of the vulva, and cervical cancer, breast cancer (small cell and ductal), penile cancer, retinoblastoma, testicular cancer, thyroid cancer, trophoblastic neoplasms, and Wilms' tumor.

It is also within the present invention that the compounds according to the invention are used as or for the manufacture of a diagnostic agent, whereby such diagnostic agent is for the diagnosis of the disorders and conditions which can be addressed by the compounds of the present invention for therapeutic purposes as disclosed herein.

For various applications, the compounds of the invention can be labelled by isotopes, fluorescence or luminescence markers, antibodies or antibody fragments, any other affinity label like nanobodies, aptamers, peptides etc., enzymes or enzyme substrates. These labelled compounds of this invention are useful for mapping the location of receptors in vivo, ex vivo, in vitro and in situ such as in tissue sections via autoradiography and as radiotracers for positron emission tomography (PET) imaging, single photon emission computerized tomography (SPECT) and the like, to characterize those receptors in living subjects or other materials. The labelled compounds according to the present invention may be used in therapy, diagnosis and other applications such as research tools in vivo and in vitro, in particular the applications disclosed herein.

### EXAMPLES

### Synthesis

### Synthesis of ethyl 16-hydroxyhexadecanoate

To a solution of 16-hydroxyhexadecanoic acid (15.00 g, 55.06 mmol) in ethanol (500 mL) was added acetyl chloride (12.97 g, 165 mmol). The solution was stirred at room temperature for 4 h, then concentrated under reduced pressure. The residue was dissolved in ethyl acetate (400 mL), and washed with sat. NaHCO₃ (3×300 mL), water (300 mL) and brine (300 mL). The organic phase was dried with NaSO₄ and concentrated under reduced pressure, affording 15.40 g (94%) of product as a white solid. ¹H NMR (400 MHz, CDCl₃): δ 4.10 (q, J=7.2 Hz, 2H), 3.60 (t, J=6.8 Hz, 2H), 2.26 (t, J=7.6 Hz, 2H), 1.65-1.50 (m, 4H), 1.40-1.20 (m, 25H).

### Synthesis of ethyl 16-azidohexadecanoate

To a solution of triphenyl phosphine (9.563 g, 36.46 mmol) in anhydrous THF (70 mL) at 0° C. was added diisopropyl azodicarboxylate (7.373 g, 36.46 mmol) dropwise. The mixture was stirred for 10 mins, then ethyl 16-hydroxyhexadecanoate (9.100 g, 30.38 mmol) in THF (40 mL) was added dropwise. After 30 mins diphenyl phosphoryl azide (10.034 g, 36.46 mmol) was added and the mixture was warmed to room temperature and stirred for 4.5 h. Water (100 mL), diethyl ether (200 mL) and brine (150 mL) was then added, and the ether layer separated and concentrated under reduced pressure. The residue was purified on silica gel by stepwise gradient elution with dichloromethane/hexane (20:80 to 100:0), yielding 8.108 g (82%) of product as a pale yellow oil. ¹H NMR (400 MHz, CDCl₃): δ 4.11 (q, J=7.2 Hz, 2H), 3.25 (t, J=6.8 Hz, 2H), 2.28 (t, J=7.6 Hz, 2H), 1.63-1.48 (m, 4H), 1.40-1.20 (m, 25H).

### Synthesis of ethyl 16-aminohexadecanoate

Ethyl 16-azidohexadecanoate (8.100 g, 24.88 mmol) and triphenyl phosphine (9.791 g, 37.33 mmol) were stirred in anhydrous THF (80 mL) at room temperature for 16 h. Water (1.792 g, 99.56 mmol) was then added, and the reaction was stirred for 16 h. The reaction was concentrated under reduced pressure and the residue was purified on silica gel by stepwise gradient elution with dichloromethane/methanol (95:5 to 40:60), yielding 4.618 g (64%) of an impure product as a beige solid. ¹H NMR (400 MHz, CDCl₃): δ 4.10 (q, J=7.2 Hz, 2H), 2.72 (m, 2H), 2.26 (t, J=7.6 Hz, 2H), 1.59 (t, J=7.2 Hz, 2H), 1.50 (t, J=7.2 Hz, 2H), 1.35-1.20 (m, 25H).

### General Procedure for the Synthesis of the Urea Moiety

To a suspension of ethyl 16-aminohexadecanoate (0.400 g, 1.33 mmol) in anhydrous THF (15 mL) under a nitrogen atmosphere was added the appropriate isocyanate (1.40 mmol). For example, in the synthesis of the ethyl ester of compound 29 (i.e. compound 30 in Table 1), the isocyanate used was 4-chloro-3-trifluoromethylphenyl isocyanate. The mixture was stirred at room temperature for 2 h, and then concentrated under reduced pressure. The residue was purified on silica gel by stepwise gradient elution with dichloromethane/ethyl acetate (100:0 to 50:50), yielding the ethyl esters as white solids.

### Synthesis of Unsaturated Analogues

Unsaturated analogues are prepared by the following method (see also Scheme 1(c) above). Step 1: 7-bromoheptanoic acid is esterified using acetyl chloride and ethanol. Step 2: cyanation using potassium cyanide and 18-crown-6 in refluxing acetonitrile. Step 3: the nitrile group is reduced to the aldehyde using Raney nickel and sodium hypophosphite in pyridine and acetic acid. Step 4: the nitrile group is reduced to the BOC-protected amine using sodium borohydride and nickel chloride. Step 5: the phosphonium compound is prepared by refluxing in toluene with triphenyl phosphine. Step 6: the unsaturated cis-bond is formed by Wittig reaction using sodium bis(trimethylsilylamide) in THF. Step 7: the amine is deprotected using p-toluenesulfonic acid. Step 8: the urea is prepared by reaction with 4-methylphenyl isocyanate in THF.

The compound numbers given below in parentheses correspond to the compound numbers in Table 1.

### Ethyl 16-[cyclohexylcarbamoyl)amino]hexadecanoate (1)

¹H NMR (400 MHz, CDCl₃): δ 4.11 (q, J=7.2 Hz, 2H), 3.45-3.55 (m, 1H), 3.13 (t, J=7.2 Hz, 2H), 2.28 (t, J=7.6 Hz, 2H), 1.94-1.90 (m, 2H), 1.72-1.68 (m, 2H), 1.63-1.51 (m, 3H), 1.47 (p, J=7.2 Hz, 2H), 1.40-1.20 (m, 27H), 1.18-1.05 (m, 3H). ¹³C NMR (100.5 MHz, CDCl₃): δ 174.0, 157.9, 60.2, 49.6, 40.9, 34.4, 33.6 (2C), 29.9, 29.6 (3C), 29.5 (2C), 29.5, 29.4, 29.3, 29.2, 29.1, 26.8, 25.4, 25.0 (2C), 24.8, 14.2.

### Ethyl 16-{[(2-phenylethyl)carbamoyl]amino}hexadecanoate (2)

¹H NMR (400 MHz, CDCl₃): δ 7.32-7.19 (m, 5H), 4.11 (q, J=7.2 Hz, 2H), 3.44 (t, J=6.8 Hz, 2H), 3.08 (t, J=7.2 Hz, 2H), 2.82 (t, J=6.8 Hz, 2H), 2.28 (t, J=7.2 Hz, 2H), 1.60 (p, J=7.2 Hz, 2H), 1.43 (p, J=7.2 Hz, 2H), 1.35-1.20 (m, 25H). ¹³C NMR (100.5 MHz, CDCl₃): δ 174.0, 158.0, 138.5, 128.8 (2C), 128.6 (2C), 126.5, 60.1, 41.9, 40.8, 36.2, 34.4, 29.9, 29.6 (3C), 29.5 (2C), 29.5, 29.4, 29.3, 29.2, 29.1, 26.8, 25.0, 14.2.

### Ethyl 16-[(tert-butylcarbamoyl)amino]hexadecanoate (4)

¹H NMR (400 MHz, CDCl₃): δ 4.11 (q, J=7.2 Hz, 2H), 3.09 (t, J=7.6 Hz, 2H), 2.28 (t, J=7.6 Hz, 2H), 1.61 (p, J=7.2 Hz, 2H), 1.43 (p, J=7.2 Hz, 2H), 1.34 (s, 9H), 1.35-1.20 (m, 25H). ¹³C NMR (100.5 MHz, CDCl₃): δ 174.0, 157.8, 60.1, 51.2, 40.8, 34.4, 29.8, 29.6 (3C), 29.6, 29.5, 29.5, 29.4 (3C), 29.4, 29.3, 29.2, 29.1, 26.9, 25.0, 14.2.

### Ethyl 16-[(phenylcarbamoyl)amino]hexadecanoate (9)

¹H NMR (400 MHz, CDCl₃): δ 7.30-7.24 (m, 4H), 7.08-7.04 (m, 1H), 4.11 (q, J=7.2 Hz, 2H), 3.20 (t, J=7.2 Hz, 2H), 2.26 (t, J=7.6 Hz, 2H), 1.60 (p, J=7.2 Hz, 2H), 1.47 (p, J=7.2 Hz, 2H), 1.35-1.20 (m, 25H). ¹³C NMR (100.5 MHz, CDCl₃): δ 174.1, 156.5, 137.8, 129.4 (2C), 124.4, 121.7 (2C), 60.2, 40.6, 40.6, 34.4, 29.9, 29.6 (3C), 29.5, 29.5, 29.5, 29.4, 29.3, 29.2, 29.1, 26.8, 25.0, 14.2.

### Ethyl 16[(benzylcarbamoyl)amino]hexadecanoate (10)

¹H NMR (400 MHz, CDCl₃): δ 7.33-7.20 (m, 5H), 4.35 (s, 2H), 4.10 (q, J=7.2 Hz, 2H), 3.12 (t, J=7.2 Hz, 2H), 2.26 (t, J=7.6 Hz, 2H), 1.59 (p, J=7.2 Hz, 2H), 1.45 (p, J=6.8 Hz, 2H), 1.35-1.20 (m, 25H). ¹³C NMR (100.5 MHz, CDCl₃): δ 173.9, 158.5, 138.3, 128.7 (2C), 127.6, 127.4 (2C), 60.2, 44.7, 40.9, 40.6, 34.4, 29.8, 29.6 (3C), 29.5, 29.5, 29.5, 29.4, 29.2, 29.2, 29.1, 26.8, 25.0, 14.2.

### Ethyl 16-{[(4-fluorophenyl)carbamoyl]amino}hexadecanoate (11)

¹H NMR (400 MHz, CDCl₃): δ 7.25-7.21 (m, 2H), 7.00-6.96 (m, 2H), 4.10 (q, J=7.2 Hz, 2H), 3.20 (t, J=7.2 Hz, 2H), 2.27 (t, J=7.6 Hz, 2H), 1.59 (p, J=7.2 Hz, 2H), 1.47 (p, J=7.2 Hz, 2H), 1.35-1.20 (m, 25H). ¹³C NMR (100.5 MHz, CDCl₃): δ 174.1, 159.7 (J_{F-C}=244 Hz), 156.3, 133.9, 123.7 (2C), 116.0 (2C), 60.2, 40.6, 34.4, 30.0, 29.6 (3C), 29.5, 29.5, 29.4, 29.3, 29.2, 29.2, 29.1, 26.8, 25.0, 14.2.

### Ethyl 16-{[(4-chlorophenyl)carbamoyl]amino}hexadecanoate (13)

¹H NMR (400 MHz, CDCl₃): δ 7.26-7.21 (m, 4H), 6.34 (s, 1H), 4.67 (t, J=6.0 Hz, 1H), 4.10 (q, J=7.2 Hz, 2H), 3.21 (q, J=6.4 Hz, 2H), 2.27 (t, J=7.6 Hz, 2H), 1.59 (p, J=7.2 Hz, 2H), 1.49 (p, J=6.8 Hz, 2H), 1.35-1.20 (m, 25H). EI-MS: m/z (%): 453.5 ([M+H]⁺).

### Ethyl 16-{[(4-methylphenyl)carbamoyl]amino}hexadecanoate (15)

¹H NMR (400 MHz, CDCl₃): δ 7.14-7.10 (m, 2H), 7.08-7.00 (m, 2H), 4.10 (q, J=7.2 Hz, 2H), 3.17 (t, J=7.2 Hz, 2H), 2.29-2.23 (m, 5H), 1.59 (p, J=7.6 Hz, 2H), 1.44 (p, J=7.2 Hz, 2H), 1.35-1.19 (m, 25H). ¹³C NMR (100.5 MHz, CDCl₃): δ 174.0, 156.6, 135.5, 133.9, 129.8 (2C), 122.0 (2C), 60.2, 40.5, 34.4, 30.0, 29.6 (3C), 29.5 (2C), 29.5, 29.4, 29.3, 29.2, 29.1, 26.9, 25.0, 20.8, 14.2.

### Ethyl 16-[(tricyclo[3.3.1.1^{3,7}]dec-1-ylcarbamoyl)amino]hexadecanoate (17)

¹H NMR (400 MHz, CDCl₃): δ 4.11 (q, J=7.2 Hz, 2H), 4.05 (t, J=6.0 Hz, 1H), 3.99 (s, 1H), 3.06 (t, J=6.8 Hz, 2H), 2.26 (t, J=7.6 Hz, 2H), 2.08-2.00 (m, 3H), 1.98-1.90 (m, 6H), 1.70-1.50 (m, 8H), 1.44 (p, J=7.2 Hz, 2H), 1.35-1.20 (m, 25H). ¹³C NMR (100.5 MHz, CDCl₃): δ 173.9, 157.1, 60.1, 50.9, 42.5 (3C), 40.5, 36.4 (3C), 34.4, 30.2, 29.6 (2C), 29.6 (3C), 29.5 (2C), 29.5 (2C), 29.4, 29.3, 29.2, 29.1, 26.9, 25.0, 14.2.

### Ethyl 16-[(ethylcarbamoyl)amino]hexadecanoate (20)

¹H NMR (400 MHz, CDCl₃): δ 4.10 (q, J=7.2 Hz, 2H), 3.18 (q, J=7.2 Hz, 2H), 3.12 (t, J=7.2 Hz, 2H), 2.26 (t, J=7.6 Hz, 2H), 1.59 (p, J=7.2 Hz, 2H), 1.47 (p, J=7.2 Hz, 2H), 1.35-1.20 (m, 25H), 1.20 (t, J=7.2 Hz, 3H). ¹³C NMR (100.5 MHz, CDCl₃): δ 174.0, 158.5, 60.1, 40.9, 35.6, 34.4, 29.9, 29.6 (3C), 29.5 (2C), 29.5, 29.5, 29.4, 29.3, 29.2, 29.1, 26.8, 25.0, 15.2, 14.2.

### Ethyl 16-{[(4-tert-butylphenyl)carbamoyl]amino}hexadecanoate (21)

¹H NMR (400 MHz, CDCl₃): δ 7.32-7.30 (m, 2H), 7.19-7.14 (m, 2H), 6.45 (s, 1H), 4.90 (t, J=5.6 Hz, 1H), 4.10 (q, J=7.2 Hz, 2H), 3.19 (q, J=6.8 Hz, 2H), 2.26 (t, J=7.6 Hz, 2H), 1.59 (p, J=7.2 Hz, 2H), 1.45 (p, J=7.2 Hz, 2H), 1.35-1.19 (m, 34H). ¹³C NMR (100.5 MHz, CDCl₃): δ 174.0, 156.2, 147.0, 135.8, 126.1 (2C), 121.4 (2C), 60.2, 40.4, 34.4, 34.3, 31.3 (3C), 30.2, 29.6 (3C), 29.5 (3C), 29.4, 29.3, 29.2, 29.1, 26.9, 25.0, 14.2.

### Ethyl 16-{[(4-methoxyphenyl)carbamoyl]amino}hexadecanoate (23)

¹H NMR (400 MHz, CDCl₃): δ 7.18-7.13 (m, 2H), 6.87-6.82 (m, 2H), 6.13 (s, 1H), 4.62 (t, J=5.6 Hz, 1H), 4.10 (q, J=7.2 Hz, 2H), 3.78 (s, 3H), 3.18 (q, J=6.8 Hz, 2H), 2.26 (t, J=7.2 Hz, 2H), 1.58 (p, J=7.2 Hz, 2H), 1.45 (p, J=6.4 Hz, 2H), 1.32-1.18 (m, 25H). ¹³C NMR (100.5 MHz, CDCl₃): δ 174.0, 157.1, 156.6, 130.8, 125.2 (2C), 114.6 (2C), 60.2, 55.5, 40.4, 34.4, 30.1, 29.6 (3C), 29.5 (3C), 29.4, 29.3, 29.2, 29.1, 26.8, 25.0, 14.2.

### Ethyl 16-{[(4-iodophenyl)carbamoyl]amino}hexadecanoate (25)

¹H NMR (400 MHz, d₆-DMSO): δ 8.30 (s, 1H), 7.48-7.45 (m, 2H), 7.18-7.15 (m, 2H), 6.00 (t, J=5.6 Hz, 1H), 4.00 (q, J=7.2 Hz, 2H), 3.01 (q, J=6.4 Hz, 2H), 2.20 (t, J=7.2 Hz, 2H), 1.50 (p, J=6.8 Hz, 2H), 1.38 (p, J=6.8 Hz, 2H), 1.30-1.18 (m, 22H), 1.13 (t, J=7.2 Hz, 2H). EI-MS: m/z (%): 545.2 ([M+H]⁺).

### Ethyl 16-{[(3,4-dichlorophenyl)carbamoyl]amino}hexadecanoate (27)

¹H NMR (400 MHz, CDCl₃): δ 7.51 (t, J=2.4 Hz, 1H), 7.29 (t, J=8.4 Hz, 1H), 7.15 (dd, J=8.4, 2.4 Hz, 1H), 6.57 (s, 1H), 4.80 (t, J=5.6 Hz, 1H), 4.10 (q, J=7.2 Hz, 2H), 3.21 (q, J=6.8 Hz, 2H), 2.27 (t, J=7.6 Hz, 2H), 1.60 (p, J=6.8 Hz, 2H), 1.49 (p, J=6.8 Hz, 2H), 1.35-1.20 (m, 25H). EI-MS: m/z (%): 487.2 ([M+H]⁺).

### Ethyl 16-({[4-chloro-3-(trifluoromethyl)phenyl]carbamoyl}amino)hexadecanoate (30)

¹H NMR (400 MHz, CDCl₃): δ 7.60 (t, J=2.8 Hz, 1H), 7.53 (dd, J=8.4, 2.8 Hz, 1H), 7.34 (t, J=8.4 Hz, 1H), 6.94 (s, 1H), 4.97 (t, J=5.6 Hz, 1H), 4.11 (q, J=7.2 Hz, 2H), 3.21 (q, J=6.8 Hz, 2H), 2.28 (t, J=7.6 Hz, 2H), 1.60 (p, J=6.8 Hz, 2H), 1.49 (p, J=7.2 Hz, 2H), 1.35-1.18 (m, 25H). ¹³C NMR (100.5 MHz, CDCl₃): δ 174.4, 155.0, 138.1, 131.9, 128.6 (q, J=31 Hz, 1C), 125.1, 123.0, 122.6 (q, J=273 Hz, 1C), 118.1, 60.3, 40.4, 34.4, 30.0, 29.4 (7C), 29.3, 29.2, 29.1, 29.0, 26.8, 25.0, 14.2.

### General Procedure for Saponification

To a solution of the ethyl ester (0.5 mmol) in ethanol (30 mL), was added 1M NaOH (10 mL). The solution was stirred at 40° C. for 3 h. The ethanol was removed under reduced pressure, and the residue was acidified with 1M HCl. The resulting suspension was filtered and the solid washed with water (10 mL) and ethanol (5 mL), yielding the carboxylic acids as white solids. For example, this procedure was used for the preparation of compound 29 from compound 30.

### 16-{[(2-phenylethyl)carbamoyl]amino}hexadecanoic acid (3)

¹H NMR (400 MHz, d₆-DMSO): δ 7.27-7.23 (m, 2H), 7.17-7.14 (m, 3H), 5.79 (t, J=6.0 Hz, 1H), 5.72 (t, J=6.0 Hz, 1H), 3.17 (q, J=6.4 Hz, 2H), 3.08 (t, J=7.2 Hz, 2H), 2.91 (q, J=6.4 Hz, 2H), 2.62 (t, J=6.4 Hz, 2H), 2.13 (t, J=7.2 Hz, 2H), 1.44 (p, J=7.2 Hz, 2H), 1.29 (p, J=6.4 Hz, 2H), 1.25-1.10 (m, 22H). ¹³C NMR (100.5 MHz, d₆-DMSO): δ 174.9, 158.4, 140.2, 129.1 (2C), 128.7 (2C), 126.4, 41.3, 36.6, 34.1, 30.4, 29.5 (3C), 29.5, 29.4, 29.4, 29.3, 29.2, 29.1, 29.0, 26.8, 25.0.

### 16-[(cyclohexylcarbamoyl)amino]hexadecanoic acid (5)

¹H NMR (400 MHz, d₆-DMSO): δ 5.56 (m, 2H), 2.91 (q, J=6.4 Hz, 2H), 2.15 (t, J=7.2 Hz, 2H), 2.75-2.65 (m, 2H), 2.65-2.55 (m, 2H), 2.55-2.40 (m, 3H), 2.35-0.95 (m, 30H). ¹³C NMR (100.5 MHz, d₆-DMSO): δ 174.9, 157.8, 48.1, 34.1, 33.8 (2C), 30.5, 29.4 (4C), 29.4 (2C), 29.3, 29.2, 29.1, 29.0, 26.8, 26.8, 25.8, 25.0 (2C).

### 16-[(phenylcarbamoyl)amino]hexadecanoic acid (6)

¹H NMR (400 MHz, d₆-DMSO): δ 9.31 (s, 1H), 7.38 (d, J=7.6 Hz, 2H), 7.13 (t, J=7.6 Hz, 2H), 7.09 (s, 1H), 6.79 (t, J=7.6 Hz, 1H), 3.00 (q, J=6.8 Hz, 2H), 2.04 (t, J=7.2 Hz, 2H), 1.43 (p, J=6.8 Hz, 2H), 1.35 (p, J=7.2 Hz, 2H), 1.30-1.10 (m, 22H). ¹³C NMR (100.5 MHz, d₆-DMSO): δ 174.8, 156.0, 141.8, 128.9 (2C), 120.8, 117.9 (2C), 34.8, 30.1, 29.1, 29.1 (3C), 29.0, 29.0, 29.0, 28.9, 28.8, 28.8, 26.8, 25.6.

### 16-[(tert-butylcarbamoyl)amino]hexadecanoic acid (7)

¹H NMR (400 MHz, d₆-DMSO): δ 5.54 (t, J=6.0 Hz, 1H), 5.51 (s, 1H), 2.88 (q, J=6.4 Hz, 2H), 2.16 (t, J=7.2 Hz, 2H), 1.46 (p, J=7.2 Hz, 2H), 1.35-1.10 (m, 33H). ¹³C NMR (100.5 MHz, d₆-DMSO): δ 175.0, 157.9, 49.3, 34.1, 30.5, 29.8 (3C), 29.5 (4C), 29.4 (2C), 29.3, 29.2, 29.1, 29.0, 26.9, 24.9.

### 16-[(benzylcarbamoyl)amino]hexadecanoic acid (8)

¹H NMR (400 MHz, d₆-DMSO): δ 7.29-7.22 (m, 2H), 7.20-7.15 (m, 3H), 6.21 (t, J=6.0 Hz, 1H), 5.85 (t, J=5.6 Hz, 1H), 4.15 (d, J=6.0 Hz, 2H), 2.95 (q, J=6.4 Hz, 2H), 2.15 (t, J=7.2 Hz, 2H), 1.44 (p, J=7.2 Hz, 2H), 1.32 (p, J=6.4 Hz, 2H), 1.30-1.10 (m, 22H). ¹³C NMR (100.5 MHz, d₆-DMSO): δ 174.8, 158.5, 141.5, 128.5 (2C), 127.4 (2C), 126.9, 43.4, 34.2, 30.4, 29.4 (3C), 29.4, 29.4, 29.3, 29.3, 29.2, 29.1, 29.0, 26.8, 24.9.

### 16-{[(4-fluorophenyl)carbamoyl]amino}hexadecanoic acid (12)

¹H NMR (400 MHz, ds-DMSO): δ 10.02 (s, 1H), 7.63 (s, 1H), 7.42 (m, 2H), 6.94 (m, 2H), 2.98 (q, J=6.8 Hz, 2H), 1.96 (t, J=7.2 Hz, 2H), 1.42-1.05 (m, 26H). EI-MS: m/z (%): 409.4 ([M+H]⁺).

### 16-{[(4-chlorophenyl)carbamoyl]amino}hexadecanoic acid (14)

1H NMR (400 MHz, d₆-DMSO): δ 9.05 (s, 1H), 7.38 (m, 2H), 7.14 (m, 2H), 6.71 (s, 1H), 3.01 (q, J=6.4 Hz, 2H), 1.99 (t, J=7.2 Hz, 2H), 1.45-1.32 (m, 4H), 1.30-1.10 (m, 22H). EI-MS: m/z (%): 425.2 ([M+H]⁺).

### 16-{[(4-methylphenyl)carbamoyl]amino}hexadecanoic acid (16)

¹H NMR (400 MHz, d₆-DMSO): δ 8.63 (s, 1H), 7.23 (m, 2H), 6.95 (m, 2H), 6.43 (s, 1H), 3.00 (q, J=6.4 Hz, 2H), 2.16 (s, 3H), 2.09 (t, J=7.2 Hz, 2H), 1.43 (p, J=6.8 Hz, 2H), 1.36 (p, J=6.8 Hz, 2H), 1.30-1.10 (m, 22H). EI-MS: m/z (%): 405.4 ([M+H]⁺).

### 16-[(tricyclo[3.3.1.1^{3,7}]dec-1-ylcarbamoyl)amino]hexadecanoic acid (18)

¹H NMR (400 MHz, d₆-DMSO): δ 5.56 (t, J=6.0 Hz, 1H), 5.40 (s, 1H), 2.87 (q, J=6.4 Hz, 2H), 2.16 (t, J=7.2 Hz, 2H), 2.00-1.90 (m, 3H), 1.85-1.75 (m, 6H), 1.60-1.50 (m, 2H), 1.46 (p, J=7.2 Hz, 2H), 1.28 (p, J=6.8 Hz, 2H), 1.25-1.10 (m, 22H). EI-MS: m/z (%): 449.4 ([M+H]⁺).

### 16-[(ethylcarbamoyl)amino]hexadecanoic acid (19)

¹H NMR (400 MHz, d₆-DMSO): δ 5.69-5.62 (m, 2H), 2.96-2.88 (m, 4H), 2.14 (t, J=7.2 Hz, 2H), 1.44 (p, J=6.8 Hz, 2H), 1.29 (p, J=6.4 Hz, 2H), 1.25-1.10 (m, 22H). ¹³C NMR (100.5 MHz, d₆-DMSO): δ 175.0, 158.4, 34.5, 34.1, 30.5, 29.5 (4C), 29.4 (2C), 29.4, 29.3, 29.2, 29.0, 26.8, 24.9, 16.2.

### 16-{[(4-tert-butylphenyl)carbamoyl]amino}hexadecanoic acid (22)

¹H NMR (400 MHz, d₆-DMSO): δ 8.24 (s, 1H), 7.22-7.13 (m, 4H), 6.01 (t, J=5.6 Hz, 1H), 2.99 (q, J=6.4 Hz, 2H), 2.12 (t, J=7.2 Hz, 2H), 1.42 (p, J=6.8 Hz, 2H), 1.36 (p, J=6.8 Hz, 2H), 1.30-1.10 (m, 31H). ¹³C NMR (100.5 MHz, d₆-DMSO): δ 175.0, 155.9, 143.8, 138.1, 125.6 (2C), 118.0 (2C), 34.1, 34.0, 31.6, 30.1, 29.4 (4C), 29.3 (2C), 29.2, 29.1, 29.0, 28.9, 26.7, 24.9.

### 16-{[(4-methoxyphenyl)carbamoyl]amino}hexadecanoic acid (24)

¹H NMR (400 MHz, d₆-DMSO): δ 8.34 (s, 1H), 7.25-7.16 (m, 2H), 6.75-6.72 (m, 2H), 6.23 (s, 1H), 3.65 (s, 3H), 3.01 (q, J=6.4 Hz, 2H), 1.96 (t, J=7.6 Hz, 2H), 1.47-1.32 (m, 2H), 1.30-1.10 (m, 22H). EI-MS: m/z (%): 421.3 ([M+H]⁺).

### 16-{[(4-iodophenyl)carbamoyl]amino}hexadecanoic acid (26)

¹H NMR (400 MHz, de-DMSO): δ 8.73 (s, 1H), 7.46-7.40 (m, 2H), 7.21-7.18 (m, 2H), 6.43 (s, 1H), 3.01 (q, J=6.4 Hz, 2H), 2.03 (t, J=7.6 Hz, 2H), 1.44 (p, J=7.2 Hz, 2H), 1.37 (p, J=7.2 Hz, 2H), 1.30-1.10 (m, 22H). EI-MS: m/z (%): 517.2 ([M+H]⁺).

### 16-{[(3,4-dichlorophenyl)carbamoyl]amino}hexadecanoic acid (28)

¹H NMR (400 MHz, de-DMSO): δ 9.11 (s, 1H), 7.77 (d, J=2.4 Hz, 1H), 7.35 (d, J=8.8 Hz, 1H), 7.19 (dd, J=8.8, 2.4 Hz, 1H), 6.61 (s, 1H), 2.99 (q, J=6.4 Hz, 2H), 2.08 (t, J=7.6 Hz, 2H), 1.50-1.35 (m, 4H), 1.30-1.10 (m, 22H). ¹³C NMR (100.5 MHz, d₆-DMSO): δ 175.9, 155.5, 141.4, 131.3, 130.7, 122.4, 119.0, 118.1, 35.0, 29.8, 29.3 (2C), 29.2 (4C), 29.2, 29.1, 29.1, 29.0, 26.6, 25.2.

### 16-({[4-chloro-3-(trifluoromethyl)phenyl]carbamoyl)amino}hexadecanoic acid (29)

¹H NMR (400 MHz, d₆-DMSO): δ 8.85 (s, 1H), 7.98 (s, 1H), 7.47 (s, 2H), 6.29 (t, J=5.6 Hz, 1H), 3.00 (q, J=6.4 Hz, 2H), 2.12 (t, J=7.6 Hz, 2H), 1.50-1.30 (m, 4H), 1.25-1.10 (m, 22H). ¹³C NMR (100.5 MHz, d₆-DMSO): δ 174.9, 155.4, 140.5, 132.3, 127.2 (q, J=31 Hz, 1C), 123.3 (q, J=273 Hz, 1C), 123.0, 122.8, 116.9, 34.1, 29.9, 29.3 (3C), 29.2 (3C), 29.1, 29.0, 29.0, 28.9, 26.6, 24.9.

### Biological Evaluation

### In Vitro Evaluation

The synthesized compounds given in Table 1 were tested for their activity in inhibiting the proliferation of MDA-MB-231 cells. This is reflected by MTT reduction, with IC₅₀ concentrations (the concentrations of the compounds required to inhibit proliferation by 50%) presented in Table 2 below.

TABLE 2

| Compound | Structure | IC₅₀ (μM) |
|---|---|---|
| 1: EUCy | 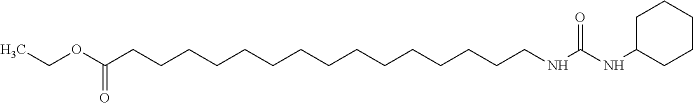 [SMILES: CCOC(=O)CCCCCCCCCCCCCCCNC(=O)NC1CCCCC1] | 0.85 ± 0.02 |
| 2: EUEPh | 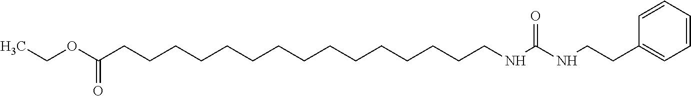 [SMILES: CCOC(=O)CCCCCCCCCCCCCCCNC(=O)NCCc1ccccc1] | 1.11 ± 0.26 |
| 3: CUEPh | 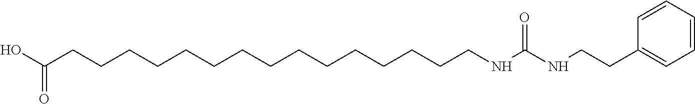 [SMILES: O=C(O)CCCCCCCCCCCCCCCNC(=O)NCCc1ccccc1] | 1.5 ± 0.08 |
| 4: EUtB | 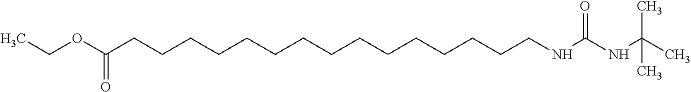 [SMILES: CCOC(=O)CCCCCCCCCCCCCCCNC(=O)NC(C)(C)C] | 6.92 ± 0.31 |
| 5: CUCy | 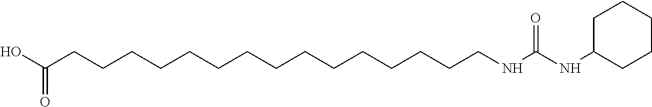 [SMILES: O=C(O)CCCCCCCCCCCCCCCNC(=O)NC1CCCCC1] | 2.78 ± 0.19 |
| 6: CUPh | 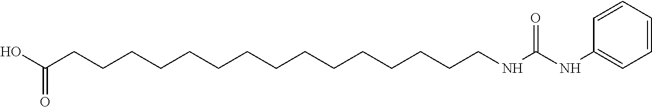 [SMILES: O=C(O)CCCCCCCCCCCCCCCNC(=O)Nc1ccccc1] | 1.2 |
| 7: CUtB | 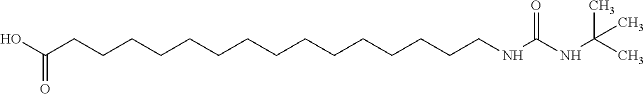 [SMILES: CC(C)(C)NC(=O)NCCCCCCCCCCCCCCCC(=O)O] | 1.22 ± 0.08 |
| 8: CUBz | 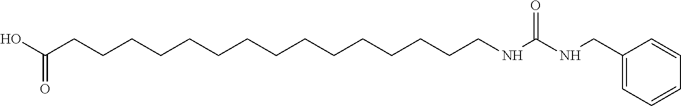 [SMILES: O=C(O)CCCCCCCCCCCCCCCNC(=O)NCc1ccccc1] | 0.72 ± 0.01 |
| 9: EUPh | 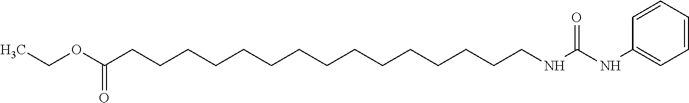 [SMILES: CCOC(=O)CCCCCCCCCCCCCCCNC(=O)Nc1ccccc1] | 1.59 ± 0.28 |
| 10: EUBz | 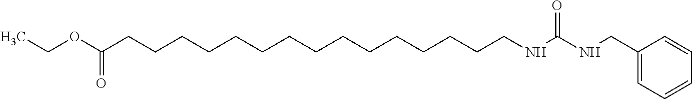 [SMILES: CCOC(=O)CCCCCCCCCCCCCCCNC(=O)NCc1ccccc1] | 0.87 ± 0.08 |
| 11: EUPhF | 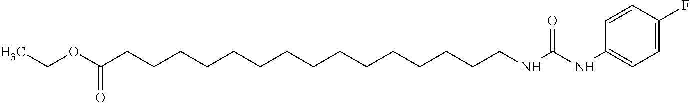 [SMILES: CCOC(=O)CCCCCCCCCCCCCCCNC(=O)Nc1ccc(F)cc1] | 0.76 ± 0.07 |
| 12: CUPhF | 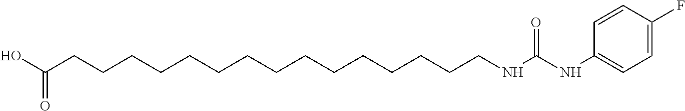 [SMILES: O=C(O)CCCCCCCCCCCCCCCNC(=O)Nc1ccc(F)cc1] | 1 |
| 13: EUPhCl | 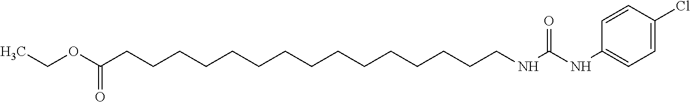 [SMILES: CCOC(=O)CCCCCCCCCCCCCCCNC(=O)Nc1ccc(Cl)cc1] | 0.08 ± 0.01 |
| 14: CUPhCl | 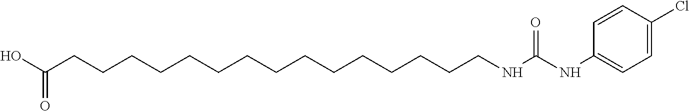 [SMILES: O=C(O)CCCCCCCCCCCCCCCNC(=O)Nc1ccc(Cl)cc1] | 0.81 |
| 15: EUT | 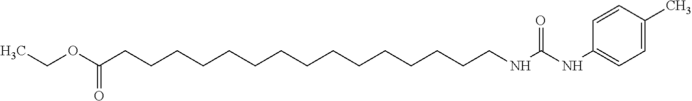 [SMILES: CCOC(=O)CCCCCCCCCCCCCCCNC(=O)Nc1ccc(C)cc1] | 0.02 ± 0.00 |
| 16: CUT | 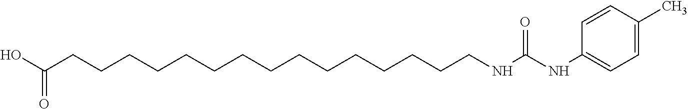 [SMILES: Cc1ccc(NC(=O)NCCCCCCCCCCCCCCCC(=O)O)cc1] | 0.75 |
| 17: EUA | 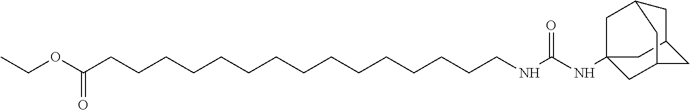 [SMILES: CCOC(=O)CCCCCCCCCCCCCCCNC(=O)NC12CC3CC(CC(C3)C1)C2] | 2.75 ± 0.27 |
| 19: CUE | 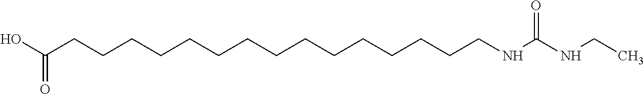 [SMILES: CCNC(=O)NCCCCCCCCCCCCCCCC(=O)O] | 1.8 |
| 20: EUE | 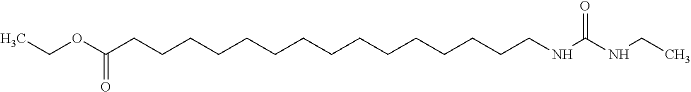 [SMILES: CCNC(=O)NCCCCCCCCCCCCCCCC(=O)OCC] | 0.66 ± 0.03 |
| 24 | 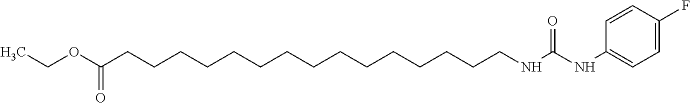 [SMILES: CCOC(=O)CCCCCCCCCCCCCCCNC(=O)Nc1ccc(F)cc1] | 1.3 |
| 26 | 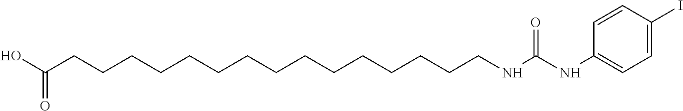 [SMILES: O=C(O)CCCCCCCCCCCCCCCNC(=O)Nc1ccc(I)cc1] | 1 |
| 28 | 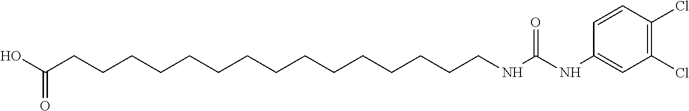 [SMILES: O=C(O)CCCCCCCCCCCCCCCNC(=O)Nc1ccc(Cl)c(Cl)c1] | 1.2 |
| 29 | 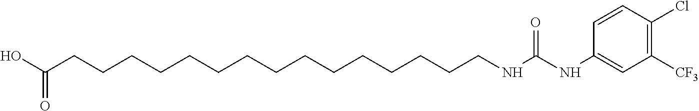 [SMILES: O=C(O)CCCCCCCCCCCCCCCNC(=O)Nc1ccc(Cl)c(C(F)(F)F)c1] | 1.1 |

Additional testing found that compound 19 also inhibited MTT reduction in MDA-MB-468 cells and MCF-7 cells, but was less potent. These are also breast cancer cell lines, but are less aggressive than the MDA-MD-231 cells.

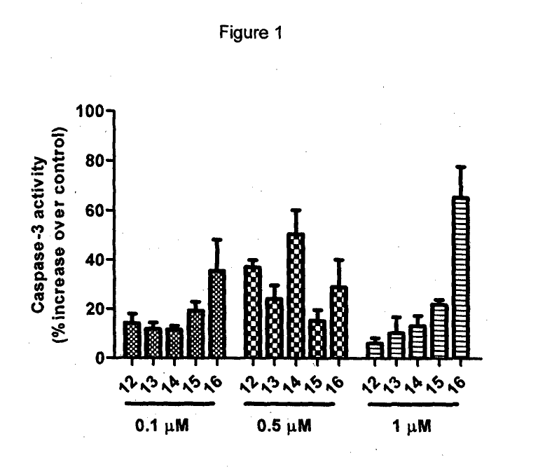

FIG. 1 shows that the compounds increased caspase-3 activity in MDA-MB-231 cells, which reflects cell killing by apoptosis. Effects of 0.1 μM compound 16 are particularly good.

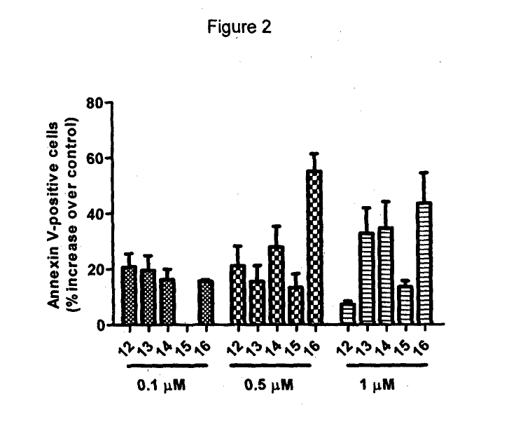

FIG. 2 shows a different apoptotic endpoint (Annexin V staining), which detects cells at early stages of apoptosis by binding to phospholipids in damaged cell membranes. Again, compound 16 increases this substantially.

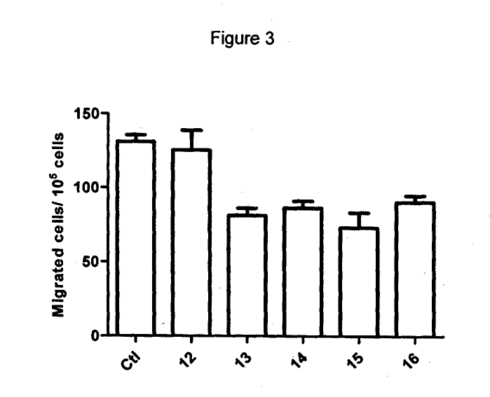

FIG. 3 is a migration assay and shows that compounds 13 to 16 are able to prevent the migration of MDA-MB-231 cells out of matrigel droplets. This supports the finding that these compounds are effective against metastatic cancer cells.

From further testing of compound 16 in matrigel assays an IC₅₀ of 3.8 μM was calculated (the concentration at which migration is inhibited to 50% of control). It was also found that compound 19 decreased migration of cells in the matrigel assay (to 61±5% of control at 10 μM).

### Experimental Details

1. Cell culture and cell treatment: Breast cancer MDA-MB-231 cells were maintained in monolayers at 37° C. in DMEM containing penicillin and streptomycin, L-glutamine and 10% fetal bovine serum in an atmosphere of 95% air and 5% CO₂. Cells were seeded into multi-well plates at 5×10⁴ cells/mL; media was replaced 24 h later with fresh serum-free DMEM containing one of the test compounds in DMSO. DMSO was added to control cells at a final concentration of 0.1%.
2. MTT assay: MDA-MB-231 cells were seeded in 96 well plates (0.2 mL/well), treated with compounds at 0.01, 0.1, 0.5, 1, 5 and 10 μM for 24 h. MTT (25 μL of 2.5 mg/mL solution) was added to each well for 2 h, after which MTT and media were removed. The blue formazan product formed from MTT by live cells was dissolved in DMSO (100 μL/well) and quantified spectrophotometrically at 540 nm in a multilabel counter. IC50s were calculated using GraphPad Prism (Prism 5.01).
3. Caspase 3 activity: MDA-MB-231 cells were seeded in 96 well plates (0.2 mL/well), and were treated with compounds at 0.1, 0.5 and 1 μM for 24 h. Caspase 3 activity was quantified using the Caspase-Glo 3/7 assay kit according to manufacturer's protocol (Promega). Briefly, after 24 h treatment, fresh serum-free media was added to wells. Cells were equilibrated at room temperature for 30 min, caspase 3/7 substrate in lysis buffer was added and the luminescence was measured. Relative caspase 3/7 activity was calculated as [(luminescence in treatment−luminescence in control)/luminescence in control×100%].
4. Annexin V assay: MDA-MB-231 cells were seeded in 6 well plates (3 mL/well), and were treated with compounds at 0.1, 0.5 and 1 μM for 24 h. The apoptotic cells were quantified by Annexin V staining according to the manufacture's protocol (BD Biosciences). Briefly, after 24 h treatment, the media and harvested cells were collected, followed by PBS washes. Cells were stained with Annexin V and PI in binding buffer for 15 min at room temperature and quantified by flow cytometry (BD Biosciences). The apoptotic cells (%) were calculated as [(Annexin V positive cells in treatment−Annexin V positive cells in control)/Annexin V positive cells in control×100%].
5. Matrigel assay: MDA-MB-231 cells were trypsinized and resuspended in serum-free DMEM media (3.5×10⁶ cells/mL). The cell suspension was mixed 1:1 with Matrigel solution (Bio Scientific). Aliquots (20 μL containing 3.5×10⁴ cells) were placed into 6-well tissue-culture dishes to form well-defined droplets and incubated at 37° C. for 5 min to enable semi-solidification. Migration media was freshly made as DMEM containing 20% fetal bovine serum, epidermal growth factor 10 pg/mL, hydrocortisone 0.4 ng/mL, vascular endothelial growth factor (VEGF, 1 pg/mL), basic fibroblast growth factor (bFGF, 20 pg/mL), insulin-like growth factor-1 (40 pg/mL), ascorbic acid (2 ng/mL) and heparin (45 ng/mL). Compounds were added in migration media (3 mL/well) and plates were then incubated for 24 h. Cells that migrated out of droplets were scored using phase-contrast microscopy and digital image analysis (Olympus).
6. Real-time RT PCR: Cell droplets from migration assays were collected, and total RNA was extracted using Tri Reagent (Astral Scientific) according to the manufacturer's protocol. RNA samples were treated with DNase (Promega) and gene expression was quantified using an Express Onestep Superscript qRT-PCT kit (Invitrogen) and gene-specific primers: heparanase (forward) GCGGTTACCCTATCCTTTTT and (reverse) GCAGCAACTTTGGCATTTC, integrin-α3 (forward) GGCCTGCCAAGCTAATGAGA and (reverse) GAGCAGCTCCATCCTCTGGTT, actin (forward) GTAGTTTCGTGGATGCCACAG and (reverse) GAGCTACGAGCTGCCTGACG. The cycling conditions (Roto-Gene™ 6000; Corbett Research) were as follows: 95° C. (15 min); 40 cycles of denaturation (95° C., 30 s); annealing (60° C., 1 min); elongation (72° C., 1 min). The gene expression was quantified by delta, delta C_{T} analysis in co-amplification reactions with actin: relative expression=2^{-ΔΔCt}, where ΔΔCt=(ΔCt_{target}−ΔCt_{actin})treated−(ΔCt_{target}−Δ Ct_{actin})control.

### In Vivo Evaluation

### Compound 15

One of the synthesised compounds given in Table 1 (compound 15, reproduced below) was tested for its activity in inhibiting the proliferation of MDA-MB-231 breast cancer cell xenografts in nude mice.

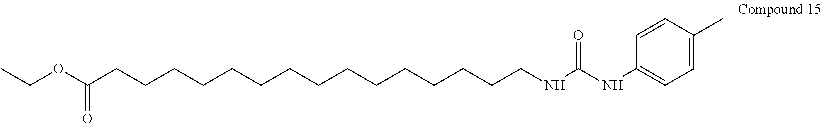 [SMILES: CCOC(=O)CCCCCCCCCCCCCCCNC(=O)Nc1ccc(C)cc1]

### Mouse Body Weight Gain or Loss is Similar in Each Group:

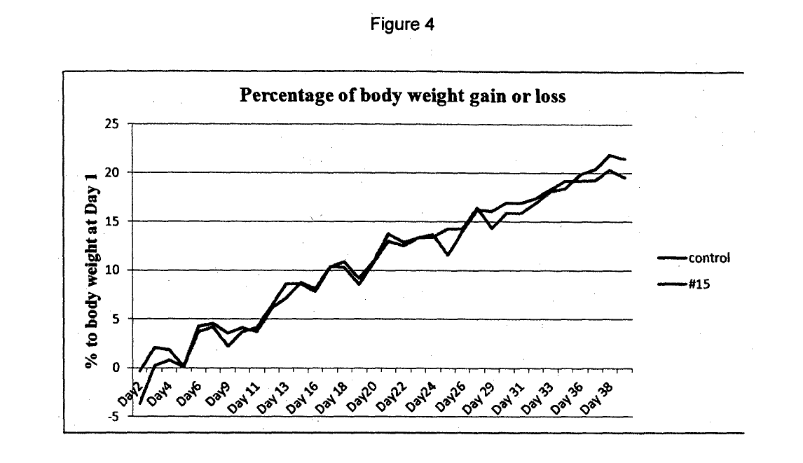

Most mice that received compound 15 or control lost weight, which was greater with compound 15 than control (FIG. 4). However, weight loss did not trigger suspension of dosing. After the first dose, weight recovered rapidly and continued at a similar rate in all animals. There was no dose limiting toxicity.

### Primary Tumour Growth:

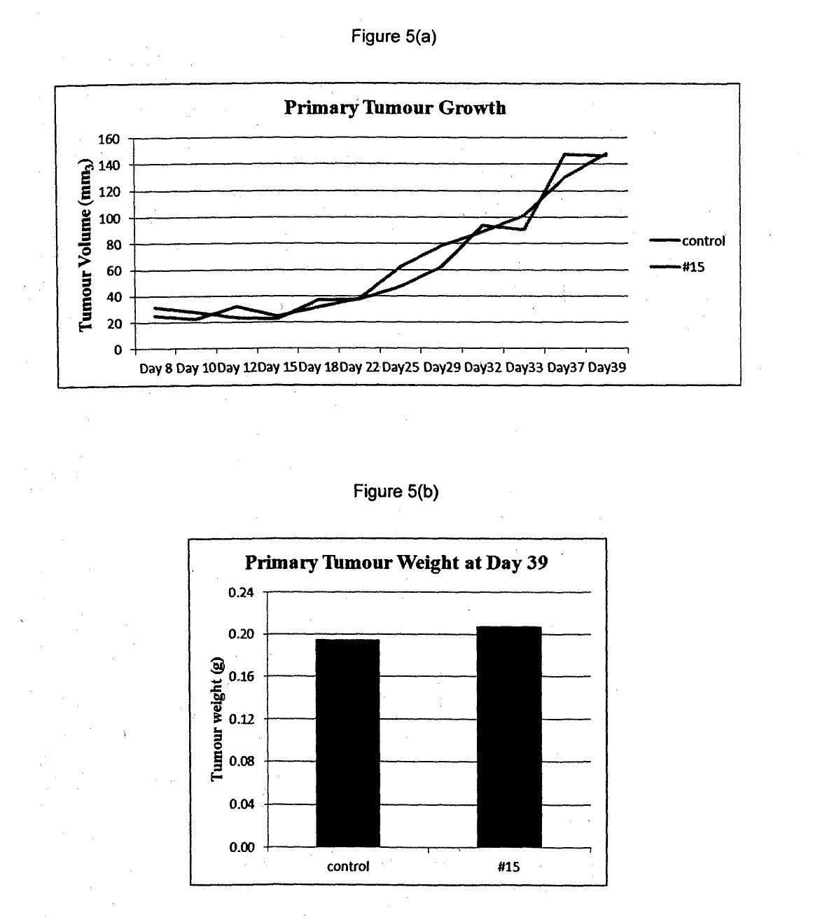

In this experimental system, tumour growth accelerated after 19 days. The growth rate and volume in treated animals was similar at all time points to the control. Final tumour weights were also similar in the two groups. Therefore compound 15 did not prevent primary tumour growth in this system, although it is presently not clear that compound 15, especially acids or derivatives of the compound 15 structure has no affect on primary tumour growth. The results of this experiment are shown in FIG. 5.

### Tumour Metastasis:

White small foci were seen in the abdominal cavities of animals in controls (7/8) but in only 1/8 mice that received compound 15; fewer foci were noted in the abdomen of that mouse compared with other groups.

### In Control Mice:

Clear oily ascites (˜2 mL) was evident in the peritoneal cavity. The livers appeared normal. Numerous small white tumour foci (˜1 mm) adhered loosely to the liver, but more (≧10) adhered tightly to the diaphragm and tissues.

In Mice Treated with Compound 15:

Clear oily ascites (in 7 mice), and milky ascites (in 1). In 7 mice the liver was well-defined, in one it appeared slightly abnormal. No foci were evident in the abdomen or on the liver, spleen or diaphragm. In one mouse there were several small foci that adhered loosely to the liver.

### Macroscopic Appearance:

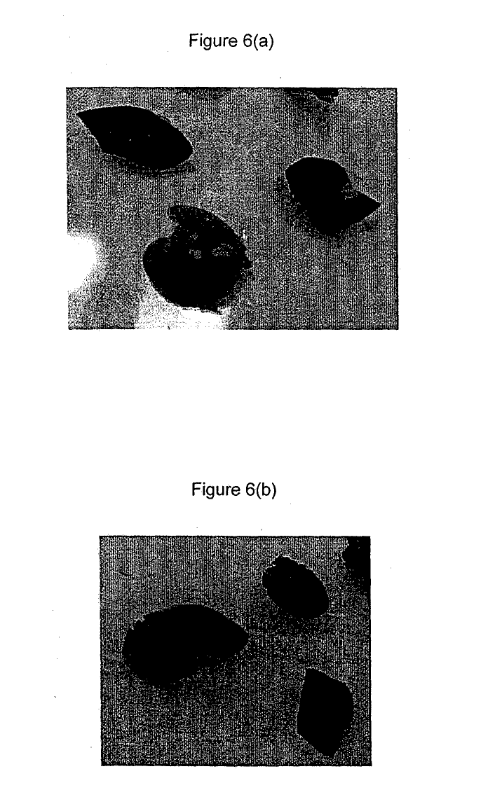

The images are shown in FIG. 6. In the control mice, white small tumour foci adhere to the surface of the liver and spleen (FIG. 6(a)). In the mice treated with compound 15, no tumour foci were evident on tissue surfaces (FIG. 6(b)).

### Histological Analysis of Primary Tumours and Tumour Foci on Tissues:

Primary tumours showed characteristic appearance in control and treated mice. The secondary tumour foci appeared to be avascular micro-metastases.

### Summary:

although the affect remains to be determined in a humansSmall white tumour foci are evident in the abdomen of control mice, but in only one of the compound 15-treated mice. Histological analysis is consistent with the white small foci being tumour micro-metastases. Compound 15 appears to suppress tumour metastasis.

### Experimental Details

### Species:

female nu/nu Balb/c mice of 5-6 weeks age at the commencement of the experiment (one week acclimatization after arrival in the animal facility).

### Experimental Design:

two groups of 8 mice each.

### Tumour Cells:

MDA-MB-231 cells, 4×10⁵ cells/100 μL (Matrigel:phosphate-buffered saline, 1:1) per mouse; subcutaneous injection into the mammary pad.

### Treatment:

4 days after cancer cell xenografting compound 15 was administered intraperitoneally at a dose of 10 mg/kg in corn oil (Sigma, containing 2% dimethylsulfoxide). Dosing continued for 6 days per week, for a total of 39 days. Control animals received 2% dimethylsulfoxide in corn oil, 100 μL per 20 g mouse for the same duration.

Body weights were measured daily and tumour sizes every three days (with calipers).

### Compound 29

One of the synthesised compounds given in Table 1 (compound 29, reproduced below) was the subject of an in vivo dose-response study in nude mice with human MDA-MB-231 breast cancer cell xenografts. One group received compound 15 in parallel.

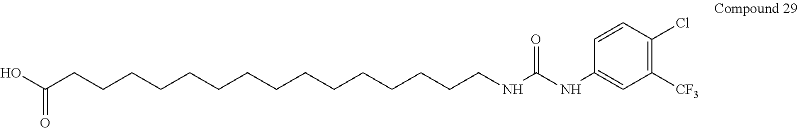 [SMILES: O=C(O)CCCCCCCCCCCCCCCNC(=O)Nc1ccc(Cl)c(C(F)(F)F)c1]

### Mouse Body Weight Gain or Loss:

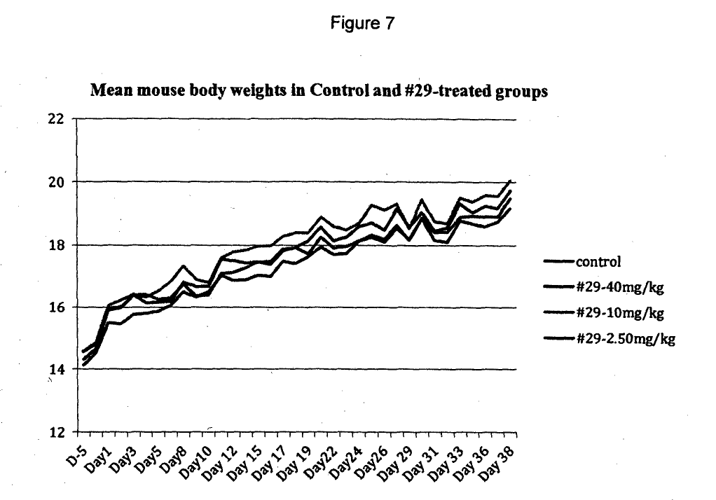

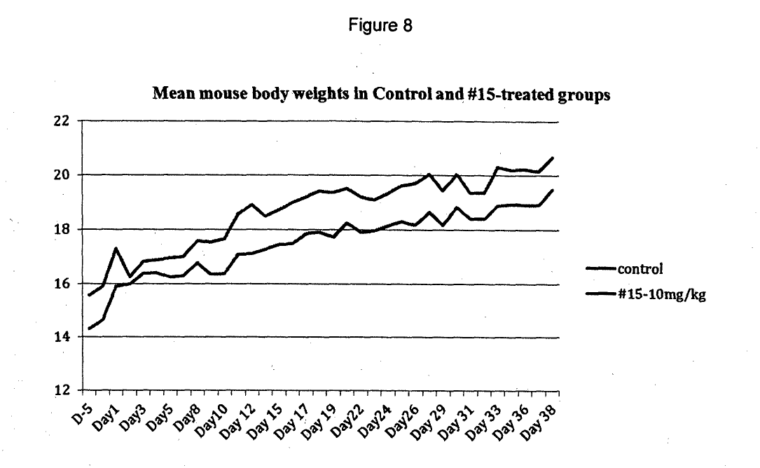

in the control, compound 29 (2.5 mg/kg), compound 29 (10 mg/kg) and compound 29 (40 mg/kg) groups, mice gained weight at a steady and similar rate during the experiment (FIG. 7). Mice in the compound 15 (10 mg/kg) group lost weight at the early stage of IP injections, but from day 8 onwards they gained weight at a rate similar to control (FIG. 8). This indicates that the treatment was non-toxic (i.e. growth rates were normal in control and compound-treated groups).

### Tumour Growth:

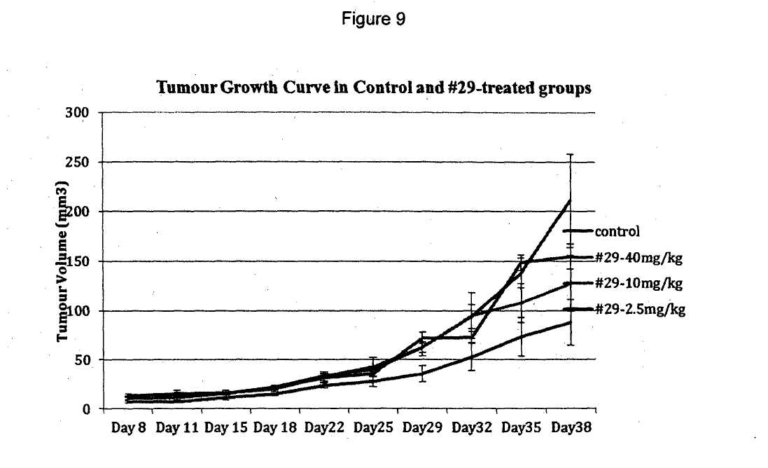

in the control and compound 29-treated groups, tumour growth was similar before day 25 (FIG. 9). At later time points, the tumour growth rate and volume in control, compound 29 (2.5 mg/kg) and compound 29 (10 mg/kg) groups were larger than the compound 29 (40 mg/kg) group. The tumour growth in the compound 29 (10 mg/kg) group decreased from day 32, and the volume was smaller than in the control and compound 29 (2.5 mg/kg) groups, but bigger than in compound 29 (40 mg/kg) group. At day 38 (the last day), the tumour volumes and weights in compound 29 (40 mg/kg) were significantly smaller than in the control and compound 29 (2.5 mg/kg) groups.

### Tumour Foci in the Peritoneal Cavity:

in the control group, tumour foci (˜1 mm) were seen in peritoneal cavities of all mice. One mouse had about 10 small foci and 4 of 5 mice had 1 to 4 foci. No tumour foci were seen in the peritoneal cavities of compound 29-treated mice.

### Proapoptotic Activity:

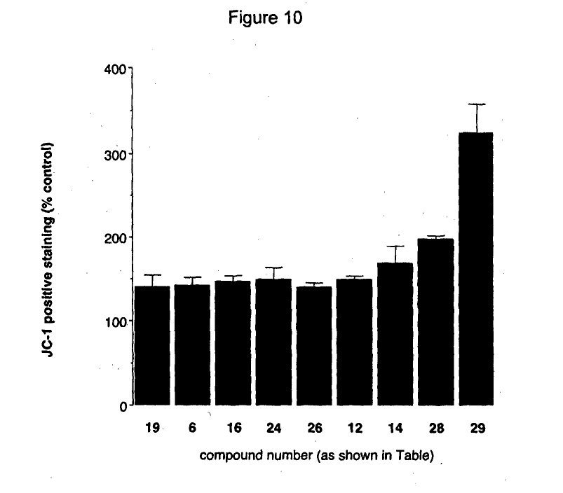

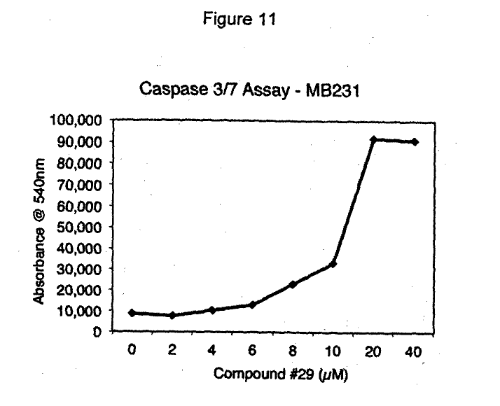

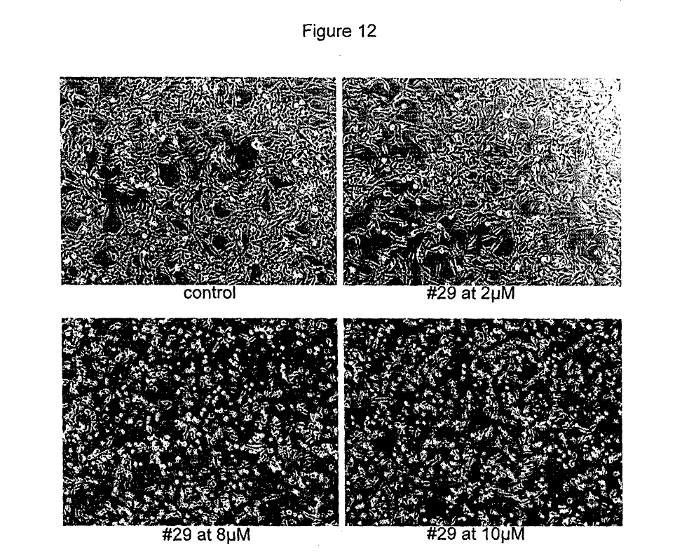

Several of the compounds were tested for the capacity to induce apoptosis in MDA-MB-231 cells (10 μM, 24 hr treatments). Increased JC-1 staining (FIG. 10) reflects mitochondrial damage consistent with apoptosis. In view of the finding with compound 29 a concentration-relationship was developed with caspase-3/7 activity (an established marker of apoptosis) as the endpoint (FIG. 11). The decreased confluence of compound 29-treated cells (FIG. 12) is consistent with cytotoxicity.

### Experimental Details

### Species:

nu/nu Balb/c; Age: 6 weeks at commencement of study; Gender: female

### Groups:

control, compound 29 (2.5 mg/kg), compound 29 (10 mg/kg), compound 29 (40 mg/kg) and compound 15 (10 mg/kg); 5 mice/group

### Tumour Cells and Xenografting:

human MDA-MB-231 cells, 4×10⁵ cells/100 μl (Matrigel:PBS 1:1)/mouse, subcutaneous injection into the 4^{th} mammary fat pad.

### Treatment:

after 4 days animals received compound 29 at 40, 10 and 2.5 mg/kg doses (in corn oil, Sigma, and 8% DMSO), or compound 15 at 10 mg/kg in corn oil at 4% DMSO, 6 days each week, for 38 days. Control: 8% DMSO in corn oil.

### Observations:

weighed six days a week; tumour sizes measured with calipers every 3 to 4 days.

### REFERENCES

Berquin I M, Edwards I J, Kridel S J, Chen Y Q. Polyunsaturated fatty acid metabolism in prostate cancer. Cancer Metastasis Rev. 2011, 30(3-4):295-309.
Chen J K, Falck J R, Reddy K M, Capdevila J, Harris R C. Epoxyeicosatrienoic acids and their sulfonimide derivatives stimulate tyrosine phosphorylation and induce mitogenesis in renal epithelial cells. J Biol Chem. 1998, 273(44):29254-61.
Inceoglu B, Schmelzer K R, Morisseau C, Jinks S L, Hammock B D. Soluble epoxide hydrolase inhibition reveals novel biological functions of epoxyeicosatrienoic acids (EETs). Prostaglandins Other Lipid Mediat. 2007, 82(1-4):42-9.

1. A compound of formula (I): 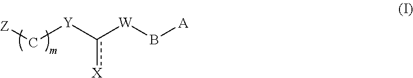
wherein
A is selected from OR¹, C(O)R¹, C(O)OR¹, C(O)NR¹R², OP(O)(OR¹)₂, C(O)OP(O)(OR¹)₂, P(OR¹)₃, C(O)OP(OR¹)₃, C(O)P(OR¹)₃, OS(O)(OR¹)₂, C(O)S(O)(OR¹)₂, OS(O)₂(OR¹), C(O)S(O)₂(OR¹), OSR¹, C(O)SR¹, OSR¹R², C(O)SR¹R², cycloalkyl, heterocycloalkyl and heteroaryl;
B is a hydrocarbon chain containing from 7 to 25 carbon atoms, wherein the hydrocarbon chain is saturated, branched or unbranched, and optionally includes one or more heteroatoms selected from O, N and S;
W and Y are selected from CH₂, O and NR¹, wherein W may form a 5- or 6-membered cycloalkyl or heterocycloalkyl ring with X and B;
X is selected from CH₂, O, NR¹ and S;
C is CH₂;
m is 0, 1 or 2;
Z is selected from alkyl, heteroalkyl, alkenyl, alkynyl, cycloalkyl, heterocycloalkyl, aryl and heteroaryl, which groups are optionally substituted,
wherein R¹ and R² are independently selected from H, OH, alkyl, heteroalkyl, alkenyl, alkynyl, cycloalkyl, heterocycloalkyl, alkylcycloalkyl, heteroalkylcycloalkyl, aryl, heteroaryl, aralkyl and heteroaralkyl, which groups are optionally substituted,
or a pharmaceutically acceptable salt, solvate or hydrate thereof.

2. The compound of formula (I) according to claim 1, wherein A is C(O)OR¹.

3. The compound of formula (I) according to claim 2, wherein R¹ is H or alkyl.

4. The compound of formula (I) according to claim 3, wherein alkyl is methyl.

5. The compound of formula (I) according to claim 3, wherein alkyl is ethyl.

6. The compound of formula (I) according to claim 1, wherein the hydrocarbon chain contains 15 carbon atoms.

7. The compound of formula (I) according to claim 1, wherein W and Y are both NH, X is O and the bond between X and the atom to which it is attached is a double bond.

8. The compound of formula (I) according to claim 1, wherein Z is a cycloalkyl group.

9. The compound of formula (I) according to claim 8, wherein the cycloalkyl group is a cyclohexyl group.

10. The compound of formula (I) according to claim 1, wherein Z is an aryl group.

11. The compound of formula (I) according to claim 10, wherein the aryl group is a phenyl group.

12. The compound of formula (I) according to claim 10, wherein the aryl group is substituted by a methyl group or a halogen.

13. The compound of formula (I) according to claim 12, wherein the halogen is fluorine or chlorine.

14. The compound of formula (I) according to claim 10, wherein the aryl group is substituted by one or more halogens, one or more alkyl groups, one or more heteroalkyl groups, or combinations thereof.

15. The compound of formula (I) according to claim 14, wherein the aryl group is substituted by a heteroalkyl group.

16. The compound of formula (I) according to claim 14, wherein the heteroalkyl group is a methoxy group.

17. The compound of formula (I) according to claim 14, wherein the aryl group is substituted by two halogens.

18. The compound of formula (I) according to claim 17, wherein the halogens are chlorine atoms.

19. The compound of formula (I) according to claim 14, wherein the aryl group is substituted by a halogen and an alkyl group.

20. The compound of formula (I) according to claim 19, wherein the alkyl group is substituted by one or more halogen atoms.

21. The compound of formula (I) according to claim 20, wherein the substituted alkyl group is CF₃.

22. The compound of formula (I) according to claim 1, wherein Z is a tert-butyl group.

23-44. (canceled)

45. A pharmaceutical composition including a therapeutically effective amount of a compound of formula (I) according to claim 1, or a mixture thereof, and one or more pharmaceutically acceptable excipients.

46. (canceled)

47. A method of treating a proliferative disorder including administering to a patient in need thereof a compound of formula (I) according to claim 1, or a mixture thereof.

48. A method of treating a proliferative disorder including administering to a patient in need thereof a pharmaceutical composition according to claim 45.

49. A method according to claim 48, wherein the proliferative disorder is a metastatic cancer.

50-52. (canceled)

53. A method of inducing apoptosis in a cell, especially a cell undergoing cell division, including contacting the cell with a compound of formula (I) according to claim 1, or a mixture thereof.

54. (canceled)

55. A method of inhibiting cell migration, including contacting the cell with a compound of formula (I) according to claim 1, or a mixture thereof.

56. (canceled)

57. A method according to claim 47, wherein the proliferative disorder is a metastatic cancer.

58. A method of inducing apoptosis in a cell, especially a cell undergoing cell division, including contacting the cell with a composition according to claim 45.

59. A method of inhibiting cell migration, including contacting the cell with a composition according to claim 45.
